(19) United States
(12) Patent Application Publication
(10) Pub. No.: US 2026/0002208 A1
(43) Pub. Date: Jan. 1, 2026

(54) CHROMOSOME INTERACTIONS

(71) Applicant: OXFORD BIODYNAMICS PLC, Oxford Oxfordshire (GB)

(72) Inventors: Aroul Selvam Ramadass, Oxford Oxfordshire (GB); Ewan Hunter, Oxford Oxfordshire (GB); Alexandre Akoulitchev, Oxford Oxfordshire (GB)

(73) Assignee: OXFORD BIODYNAMICS PLC, Oxford Oxfordshire (GB)

(21) Appl. No.: 18/259,908

(22) PCT Filed: Jan. 6, 2022
(86) PCT No.: PCT/GB2022/050009

§ 371 Date: Jun. 29, 2023

Related U.S. Application Data
(60) Provisional application No. 63/134,776, filed on Jan. 7, 2021.
(60) Provisional application No. 63/134,765, filed on Jan. 7, 2021.
(60) Provisional application No. 63/163,700, filed on Mar. 19, 2021.
(60) Provisional application No. 63/163,698, filed on Mar. 19, 2021.

(51) Int. Cl. C12Q 1/6876; C12Q 1/48; C12Q 1/6806; C12Q 1/6827; C12Q 1/6851; C12Q 1/686

(52) U.S. Cl. CPC C12Q 1/6876; C12Q 1/48; C12Q 1/6806; C12Q 1/6827; C12Q 1/6851; C12Q 1/686; C12Y 207/07; C12Q 2600/106; C12Q 2600/118

(57) ABSTRACT

A process for analysing chromosome interactions relating to coronavirus infection.

### CROSS-REFERENCE

This application is a 371 National Stage filing and claims the benefit under 35 U.S.C. § 120 of International Application No. PCT/BGB2022/050009, filed 6 Jan. 2022, which claims priority to U.S. Provisional Application No. 63/134,765, filed 7 Jan. 2021; U.S. Provisional Application No. 63/163,698 filed 19 Mar. 2021; U.S. Provisional Application No. 63/134,776 filed 7 Jan. 2021; and U.S. Provisional Application No. 63/163,700 filed 19 Mar. 2021; each of which is incorporated herein by reference in its entirety.

### SEQUENCE LISTING INCORPORATION BY REFERENCE

The application herein incorporates by reference in its entirety the sequence listing material in the ASCII text file named “Corrected 12440_005US1_seq_listing_2_ST25.txt”, created Jul. 23, 2025, and having the size of 798,720 bytes.

### FIELD OF THE INVENTION

The invention relates to infectious disease processes.

### BACKGROUND OF THE INVENTION

Coronaviruses are a group of related RNA viruses that cause diseases in mammals and birds. In humans and birds, they cause respiratory tract infections that can range from mild to lethal. Mild illnesses in humans include some cases of the common cold, but there are more lethal varieties such as Covid-19.

### SUMMARY OF THE INVENTION

The inventors have identified chromosome conformation signatures relevant to coronavirus infection prognosis, and in particular to Covid-19 infection prognosis. This allows stratification of patients to identify prognostically in advance high-risk individuals who will progress to severe deterioration leading to the need for ICU (intensive care unit) support when they are exposed to coronavirus, and in particular to Covid-19. The decision to place a patient in ICU is based largely on the individual situation of the patient, and is normally done when there is clear clinical manifestation of complications which are not responding to clinical standards of care. Coronavirus complications, in all their wide manifestations, are linked to hyperinflammation, and immune overreaction targeting individual organs. The stable 3D genomic systemic profile analysed by the inventors carries strong prognostic information, discriminating asymptomatic, mild and severe (ICU) outcomes to lasting coronavirus infections before the outcomes manifested themselves.

Accordingly, the invention provides a method of detecting prognosis for coronavirus infection in an individual, comprising determining the presence or absence of one or more chromosome interactions represented by the probes shown in Table 1 or 3, to thereby determine said prognosis in the individual. The invention also provides a method of detecting prognosis for coronavirus infection in an individual comprising determining the presence or absence of one or more chromosome interactions represented by the probes shown in Table 7, to thereby determine said prognosis in the individual. The invention further provides a method of detecting prognosis for coronavirus infection in an individual comprising determining the presence or absence of one or more chromosome interactions represented by the probes shown in any of Tables 8, 9, 10 and 11, to thereby determine said prognosis in the individual.

The invention provides a method of determining prognosis for coronavirus infection in an individual comprising determining the presence or absence of one or more chromosome interactions represented by the probes shown in any of Tables 14, 15, 6 and 17, to thereby determine said prognosis in the individual.

The invention also provides a method of detecting the presence of, or susceptibility to sepsis, in an individual, comprising determining the presence or absence of one or more chromosome interactions represented by the probes shown in Table 12 or 13.

Preferably the method is carried out to select an individual for receiving therapy or a treatment. The method may be carried out on individual that has been preselected, for example, based on a physical characteristic, risk factor or the presence of a symptom.

### BRIEF DESCRIPTION OF THE DRAWINGS

FIG. 1 shows a preferred method for carrying out the marker detection step of the invention.

FIG. 2 shows leucocyte lineage.

FIG. 3 shows the study design.

FIG. 4 shows a PCA for 38 patients (from 3 cohorts) for asymptomatic (square), mild (triangle) and ICU (severe) (circle).

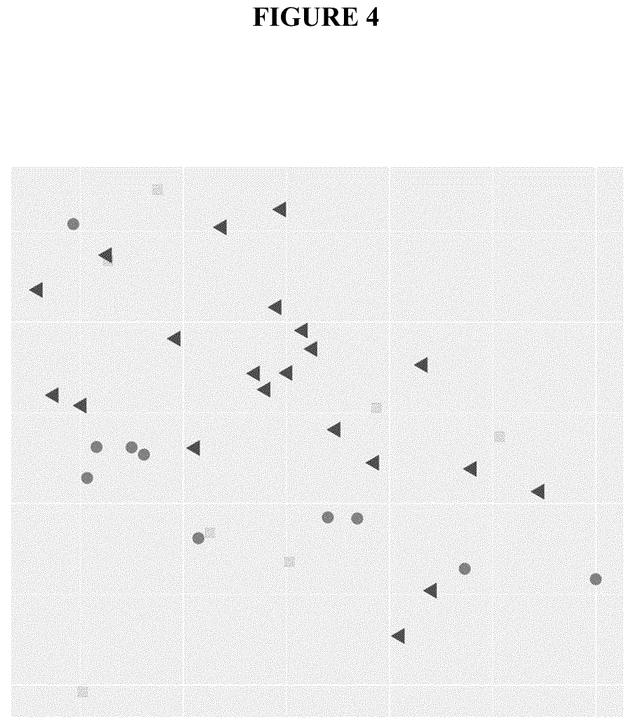

FIG. 5 shows a PCA only for mild and (circle) ICU (square).

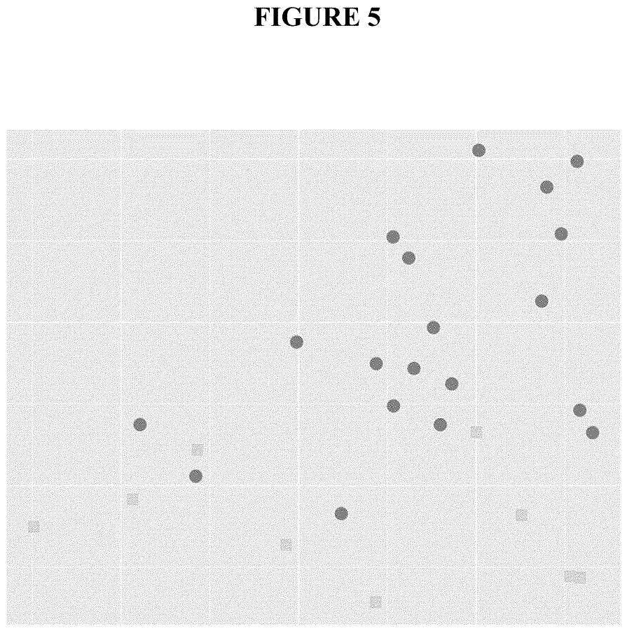

FIG. 6 shows the analytical pipeline.

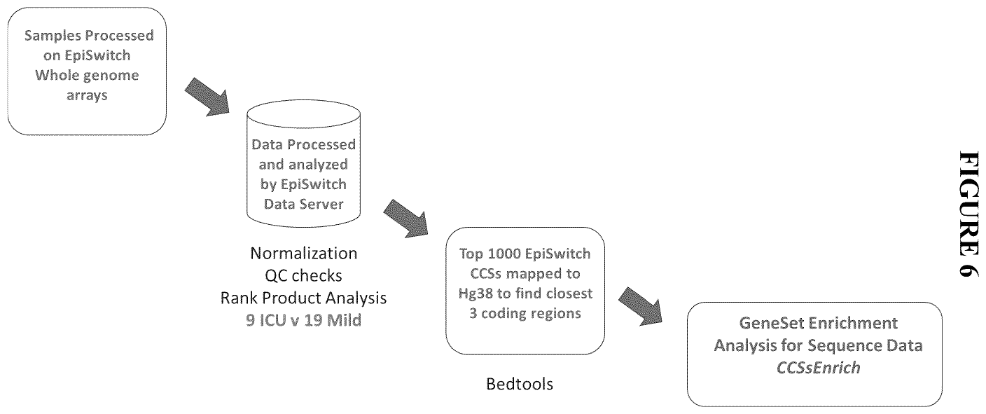

FIG. 7 shows 20 Hallmark GeneSets for ICU versus mild. The central column represents GeneSets shared between ICU and mild. The four sets on the bottom right indicated immune processes associated with ICU patients. These 20 gene sets are identified on the basis of the top EpiSwitch markers and their localisation at the gene position is described by GeneSets.

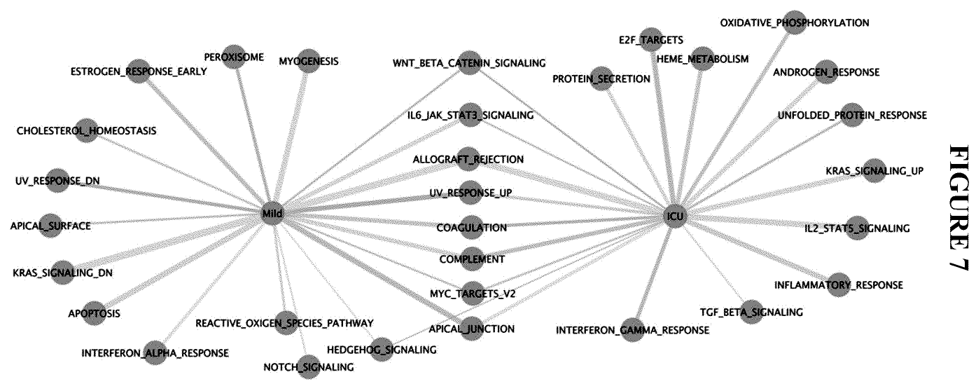

FIG. 8 shows BioCarta pathways for ICU versus mild. Shared gene sets are shown in the centre and the far left 7 gene sets show immune processes associated with ICU. This analysis is based on the genomic positions of the top EpiSwitch markers to see which overlapping genes are part of which pathways.

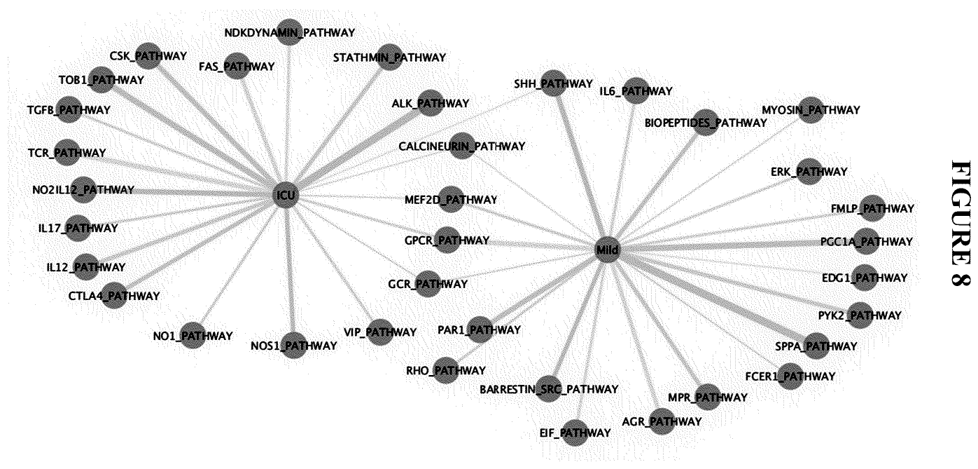

FIG. 9 shows top 20 reactome pathways for ICU versus mild. The 7 gene sets on the right of ICU relate to immune processes. Immune and angiotensin processes are shown in the mild. In this analysis the EpiSwitch positions were compared to the same genomic positions described in the reactome database.

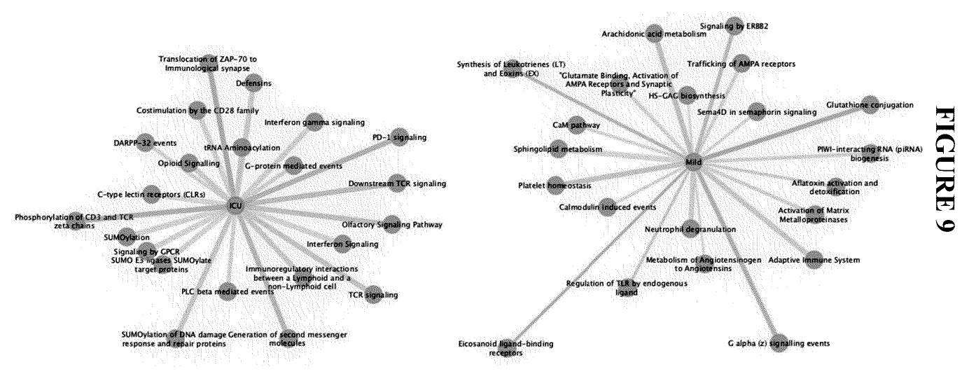

FIG. 10 shows the top 100 significant markers for mild associated to immune processes. This is not a two dimensional PCA, but a similar single dimensional standard Linear Discriminant Analysis (all complexity reduce to one linear score for three clinical outcomes/phenotypes). It is produced on the basis of the top 100 significant markers, present only in mild cases and not asymptomatic or severe ICU, and overlapping in their positions with immuno-genetic loci in the genome (hence called Immune EpiSwitch markers). This analysis contains 80 patients: on top of three cohorts from UK (1) and USA (2), and has 42 patients from Lima, Peru, all collected at the time when Lima became the site of the highest fatalities from Covid-19. The top of the diagram is to the left of the page. The top set of circles is severe (bottom left of the page). The middle set of circles is mild (top of the page). The bottom set of circles is asymptomatic (to the middle right of the page).

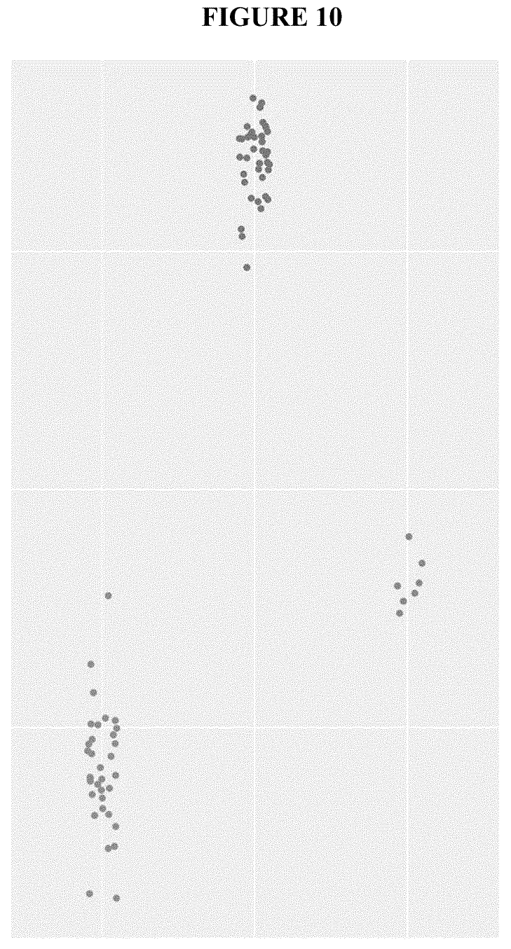

FIG. 11 shows a genome view of the markers of FIG. 10.

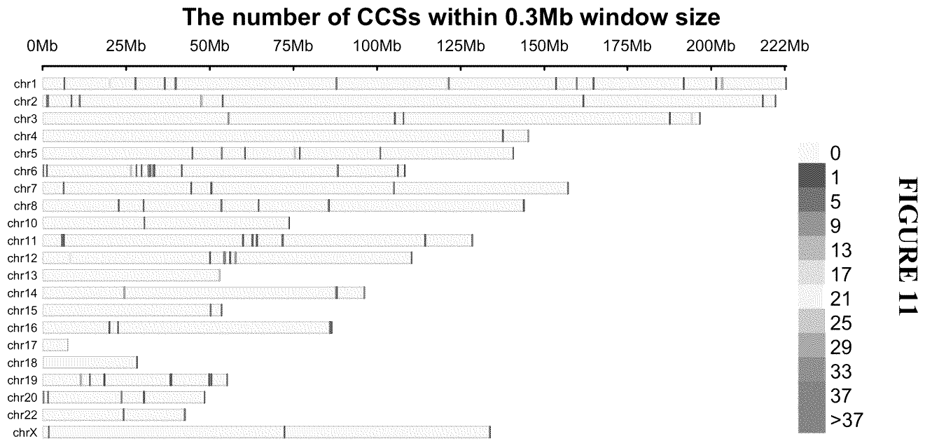

FIG. 12 shows the top 100 significant markers for ICU that are associated with immune processes, showing ICU is a distinct phenotype. This analysis is done in the same way as for FIG. 10, except the top markers that were used were all statistically significant and present in ICU (severe) group of patients, not in the mild or asymptomatic. This demonstrates that on the basis of top markers unique to ICU outcomes and present in advance of complications at the time of blood collection and first Covid-19 testing, one can prognostically identify and distinguish the profile present for severe outcome, as a distinct phenotype in 3D genomics associated with a distinct clinical outcome. The top of the diagram is to the left of the page. The top set of circles is severe (top left of the page). The middle set of circles is mild (second set of circles from the bottom of the page). The bottom set of circles is asymptomatic (to the bottom right of the page).

FIG. 13 shows a genome view of the markers of FIG. 12.

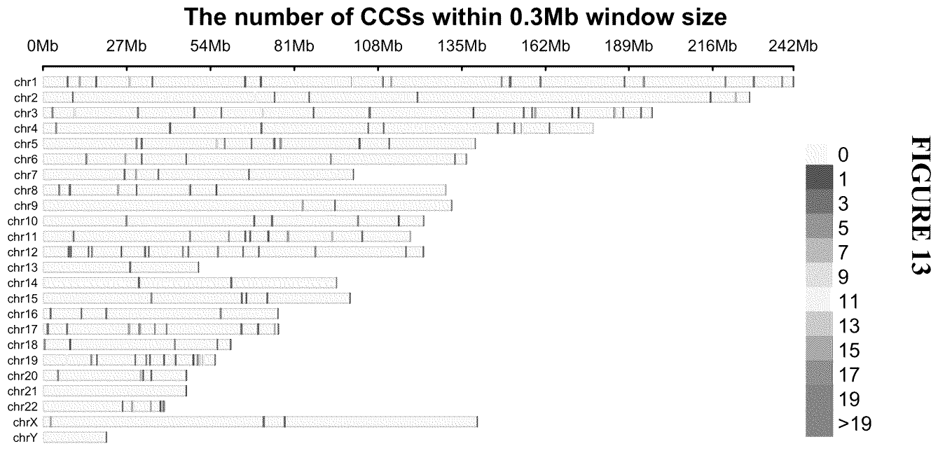

FIG. 14 relates to the how the top 50 ICU markers associated with immune processes were selected for LDA analysis and classification statistics. The markers were selected using a 30 training sample set and then used to classify the 12 test set.

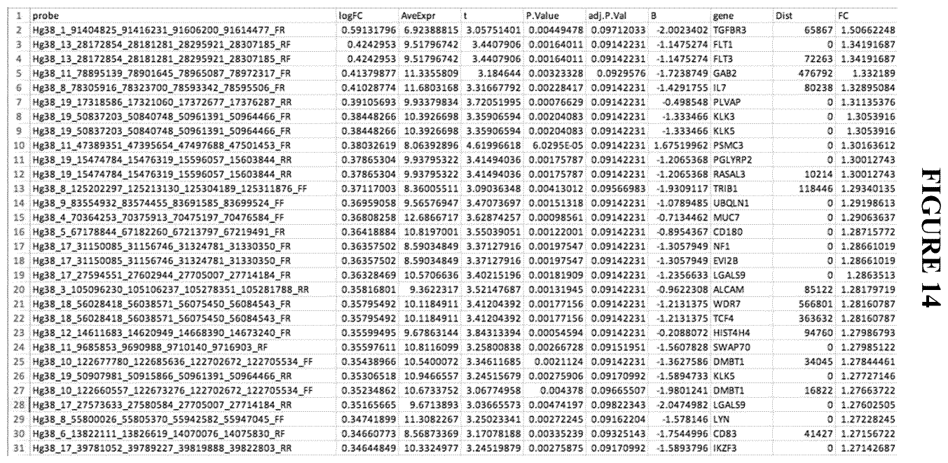

FIG. 15 shows enrichment of pathways using the genetic location enriched with the top 50 markers.

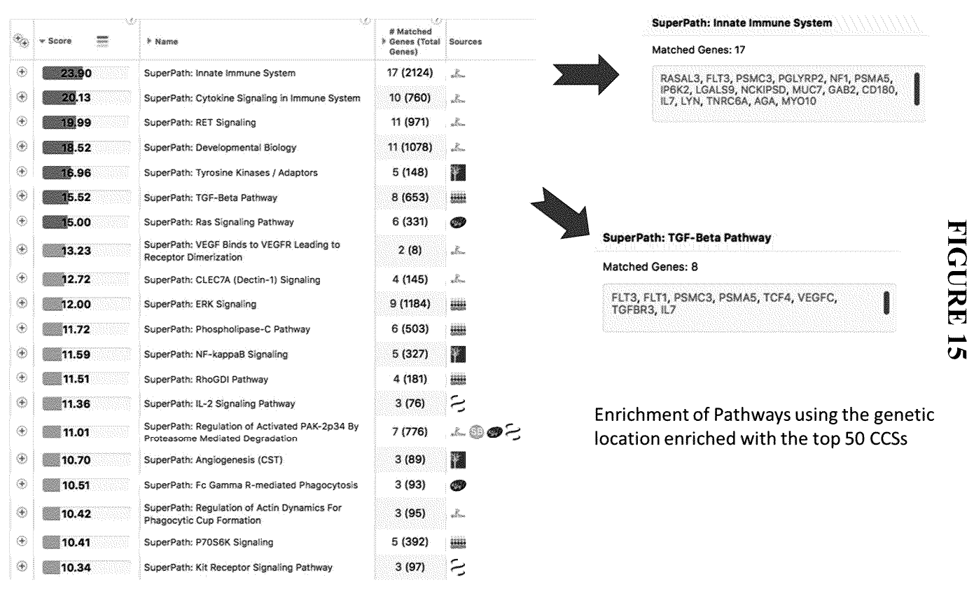

FIG. 16 shows enrichment of compounds using the genetic location enriched with the top 50 markers.

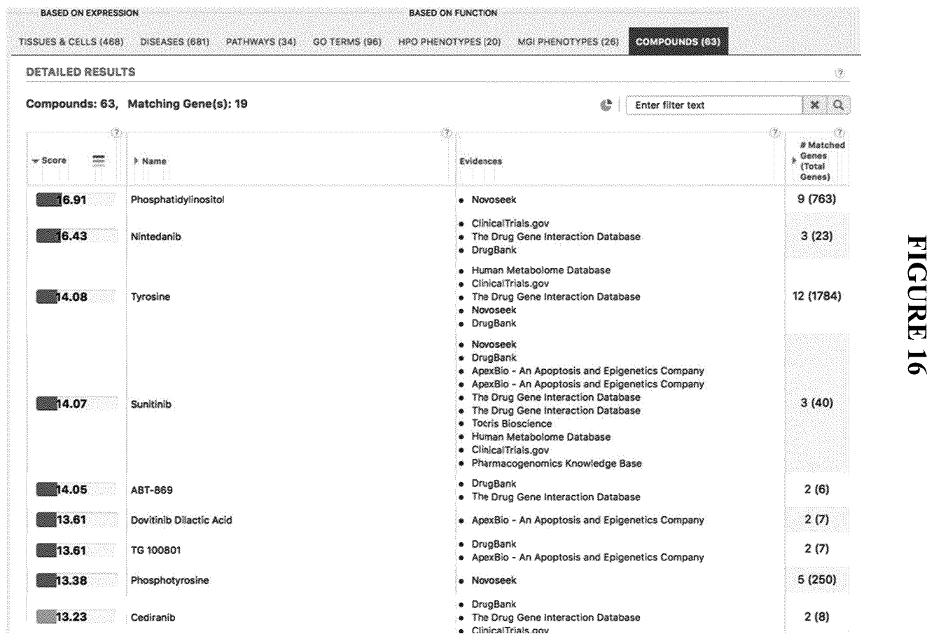

FIG. 17 shows the LDA plot of the 30 training set using the top 50 markers. The left of the page is the top of the diagram. The triangles at the top are severe (top left of the page). The circles at the bottom are mild (bottom right of the page).

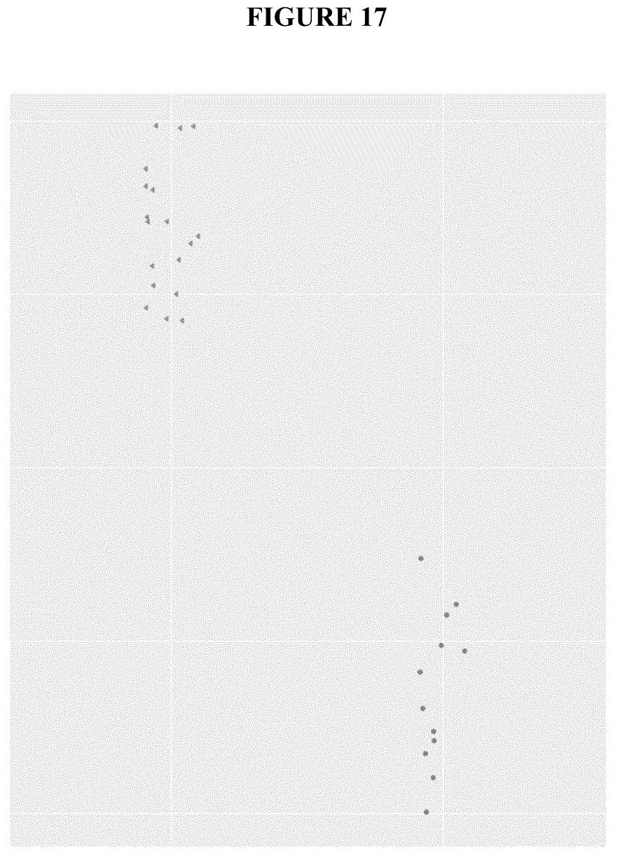

FIG. 18 shows the LDA plot of the 30 training set with the 12 set test.

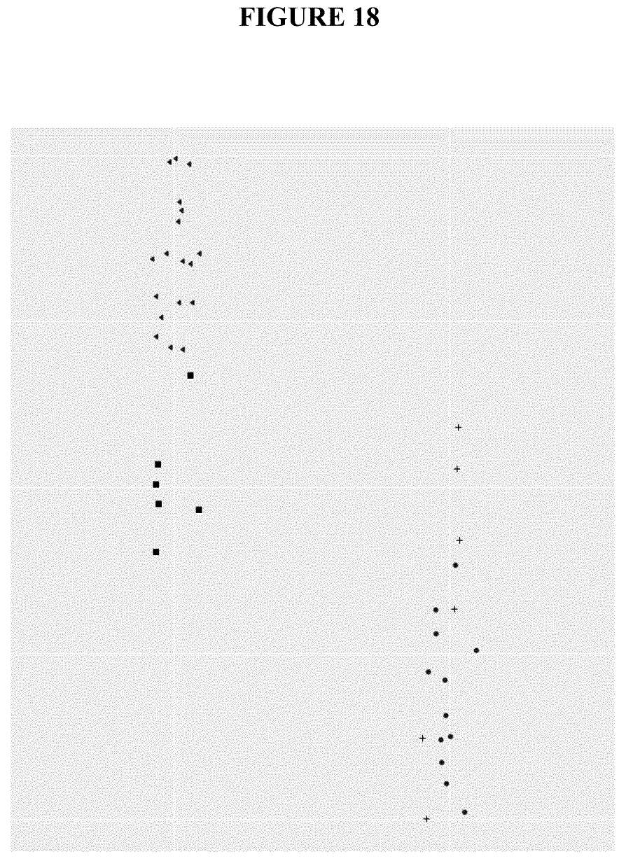

FIG. 19 shows patient calls by LDA on 42 patients from the Lima cohort and the efficacy of prognostic stratification by LDA calls.

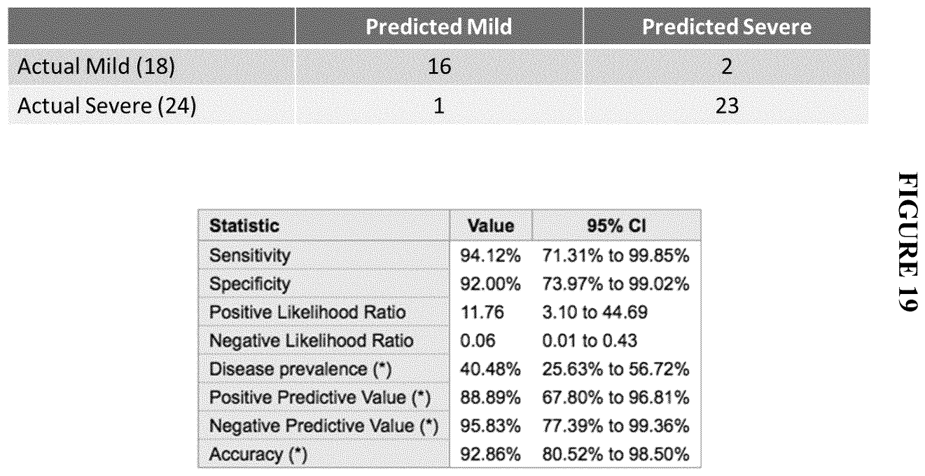

FIG. 20 shows a standard STRING network analysis-functional protein association networks, where the immune genes that have ICU/Severe COVID associated EpiSwitch significant markers are overimposed onto the known interaction networks. It matches the known network very well and shows a highly connected part of the regulatory network through EpiSwitch dysregulated genes. No additional nodes form outside of Episwitch list had to be added for completion of the network. Table 5 shows the names of the key genes which are involved and provides further data.

FIG. 21 shows how Table 6 is to be interpreted.

FIG. 22 shows pathways which relate to both Covid infection and sepsis identified by analysis of the markers found in the present work. In particular PSMA5 is implicated, and HG38_1_109341939_109348573_109359719_109366704_RR (OBD183_q481.q483) is a shared marker. PSMA5, CD3D and CD3G are matched genes in the pathway relating to antigen processing-cross presentation and KLRG1, CD3D and CD3G are matched genes in the pathway related to immunoregulatory interactions between a lymphoid and non-lymphoid cell.

### DETAILED DESCRIPTION OF THE INVENTION

### Terms Used Herein

The method of the invention may be referred to as the ‘process’ of the invention herein.

The chromosome interactions which are typed may be referred to as ‘markers’, ‘CCS’, ‘chromosome conformation signature’, ‘epigenetic interaction’ or ‘EpiSwitch markers’ herein.

The word ‘type’ will be interpreted as per the context, but will usually refer to detection of whether a specific chromosome interaction is present or absent.

### The Epigenetic Interactions Relevant to the Invention

The chromosome interactions which are typed in the invention are typically interactions between distal regions of a chromosome, said interactions being dynamic and altering, forming or breaking depending upon the state of the region of the chromosome. That state will reflect different aspects of coronavirus infection and therefore the invention can be carried out to detect the prognosis for the infection, and in particular to detect susceptibility to severe disease (which may for example be characterised by any of the detrimental effects of the immune response mentioned herein).

The chromosome interaction may, for example, reflect if it is being transcribed or repressed. Chromosome interactions which are specific to coronavirus infection subgroups as defined herein have been found to be stable, thus providing a reliable means of measuring the differences between the two subgroups (for example reflecting different outcomes of the infection).

Chromosome interactions specific to coronavirus infection will normally occur early in the disease process, for example compared to other epigenetic markers such as methylation or changes to binding of histone proteins. Thus the process of the invention is able to detect disease at an early stage. This allows early intervention (for example treatment) which as a consequence will be more effective. Chromosome interactions also reflect the current state of the individual and therefore can be used to assess changes to disease status. Furthermore there is little variation in the relevant chromosome interactions between individuals within the same subgroup. Detecting chromosome interactions is highly informative with up to 50 different possible interactions per gene, and so processes of the invention can for example interrogate 500,000 possible different interactions.

Chromosomal interactions may overlap and include the regions of chromosomes shown to encode relevant or undescribed genes, but equally may be in intergenic regions. It should further be noted that the inventors have discovered that chromosome interactions in all regions are equally important in determining the status of a chromosomal locus.

The chromosome interactions which are detected in the invention could be impacted by changes to the underlying DNA sequence, by environmental factors, DNA methylation, non-coding antisense RNA transcripts, non-mutagenic carcinogens, histone modifications, chromatin remodelling and specific local DNA interactions. However it must be borne in mind that chromosome interactions as defined herein are a regulatory modality in their own right and do not have a one to one correspondence with any genetic marker (DNA sequence change) or any other epigenetic marker.

The chromosome interaction which is detected in the method of the invention can be in any gene, chromosome region defined in the tables, or in any pathway shown herein (for example in any gene in such a pathway).

Chromosome interactions may be impacted by changes to the underlying nucleic acid sequence which themselves do not directly affect a gene product or the mode of gene expression. Such changes may be for example, SNPs within and/or outside of the genes, gene fusions and/or deletions of intergenic DNA, microRNA, and non-coding RNA. For example, it is known that roughly 20% of SNPs are in non-coding regions, and therefore the process as described is also informative in non-coding situation. In one aspect the regions of the chromosome which come together to form the interaction are less than 5 kb, 3 kb, 1 kb, 500 base pairs or 200 base pairs apart on the same chromosome.

The chromosome interaction which is detected may be within a gene, such as any gene mentioned herein. However it may also be upstream or downstream of the gene, for example up to 50,000, up to 30,000, up to 20,000, up to 10,000 or up to 5000 bases upstream or downstream from the gene or from the coding sequence.

### The Process of the Invention

The process of the invention comprises a typing system for detecting chromosome interactions relevant to coronavirus infection prognosis. Any suitable typing method can be used, for example a method in which the proximity of the chromosomes in the interaction is detected. The typing method may be performed using the EpiSwitch™ system mentioned herein which for example may be carried out by a method comprising the following steps (for example on a sample from the subject):
(i) cross-linking regions of chromosome which have come together in a chromosome interaction,
(ii) optionally isolating the cross-linked DNA from said chromosomal locus
(iii) subjecting the cross-linked DNA to cleavage, and
(iv) ligating the nucleic acids present in the cross-linked entity to derive a ligated nucleic acid with sequence from both the regions which formed a chromosomal interaction.

Detection of this ligated nucleic acid allows determination of the presence or absence of a particular chromosome interaction. The ligated nucleic acid therefore acts as a marker for the presence of the chromosome interaction. Preferably the ligated nucleic acid is detected by PCR or a probe based method, including a qPCR method.

In the method the chromosomes can be cross-linked by any suitable means, for example by a cross-linking agent, which is typically a chemical compound. In a preferred aspect, the interactions are cross-linked using formaldehyde, but may also be cross-linked by any aldehyde, or D-Biotinoyl-e-aminocaproic acid-N-hydroxysuccinimide ester or Digoxigenin-3-O-methylcarbonyl-e-aminocaproic acid-N-hydroxysuccinimide ester. Para-formaldehyde can cross link DNA chains which are 4 Angstroms apart. Preferably the chromosome interactions are on the same chromosome. Typically the chromosome interactions are 2 to 10 Angstroms apart.

The cross-linking is preferably in vitro. The cleaving is preferably by restriction digestion with an enzyme, such as TaqI. The ligating may form DNA loops.

Where PCR (polymerase chain reaction) is used to detect or identify the ligated nucleic acid, the size of the PCR product produced may be indicative of the specific chromosome interaction which is present, and may therefore be used to identify the status of the locus. In preferred aspects the primers shown in any table herein are used, for example the primer pairs shown in Table 1 or 3 are used (corresponding to the chromosome interaction which is being detected). In other preferred aspects the primers shown in Table 8, 9, 10, 11, 12, 13, 14, 15, 16 or 17 are used (corresponding to the chromosome interaction which is being detected). Homologues of such primers or primer pairs may also be used, which can have at least 70% identity to the original sequence.

Where a probe is used to detect or identify the ligated nucleic acid this is generally by Watson-Crick based base-pairing between the probe and ligated nucleic acid. Probe sequences as shown in any table herein may be used, for example the probe sequences shown in Table 1 or 3 (corresponding to the chromosome interaction which is being detected). Probe sequences as shown in Table 8, 9, 10, 11, 12, 13 and 14 may used (corresponding to the chromosome interaction which is being detected). Homologues of such probe sequences may also be used, which can have at least 70% identity to the original sequence.

Typing according to the process of the invention may be carried out at multiple time points, for example to monitor the progression of the disease. This may be at one or more defined time points, for example at at least 1, 2, 5, 8 or 10 different time points. The durations between at least 1, 2, 5 or 8 of the time points may be at least 5, 10, 20, 50, 80 or 100 days. Typically there are 3 time points at least 50 days apart.

### Subgroups and Personalised Treatment

As used herein, a “subgroup” preferably refers to a population subgroup, more preferably a subgroup in the population of a particular organism such as a particular eukaryote, animal, bird or mammal. Most preferably, a “subgroup” refers to a subgroup in the human population. Therefore the process of the invention is preferably carried out to detect the presence of coronavirus infection in a eukaryote, such as an animal, mammal or bird, and preferably in a human. The process of the invention may be carried out for diagnostic or prognostic purposes.

The invention includes detecting and treating particular subgroups in a population, typically differing int their prognosis to coronavirus infection. The inventors have discovered that chromosome interactions differ between subsets (for example at least two subsets) in the relevant population. Identifying these differences will allow physicians to categorize their patients as a part of one subset of the population. The invention therefore provides physicians with a process of personalizing medicine for the patient based on their epigenetic chromosome interactions. Such testing may be used to select how to subsequently treat the patient, for example the type of drug and/or its dose and/or its frequency of administration.

The Individual that is Tested

The individual that is tested in the process of the invention may have been selected in some way, for example based on a risk factor or physical characteristic. The individual may have been selected based on being symptomless for a given disease, or being in the early stages of the disease or having a mild form of the disease.

The individual may be susceptible to any condition mentioned herein and/or may be in need of any therapy mentioned in. The individual may be receiving any therapy mentioned herein. In particular, the individual may have, or be suspected of having, coronavirus infection. The individual may have, or be suspected of having, Covid-19 infection. Thus the invention includes a process of typing a patient to determine coronavirus prognosis, which is equivalent to determining the subgroup they belong to.

The individual may have one or more of the following characteristics:
having received an organ transplant
having received chemotherapy or antibody treatment for cancer, including immunotherapy
having received intense course of radiotherapy (radical radiotherapy) for lung cancer
having received targeted cancer treatments that can affect the immune system (such as protein kinase inhibitors or PARP inhibitors)
they have, or have had, blood or bone marrow cancer (such as leukaemia, lymphoma or myeloma)
they have, or have had, a severe lung condition (such as cystic fibrosis, severe asthma or severe COPD)
a high risk of getting infections (such as SCID or sickle cell)
they are taking medicine that makes individuals much more likely to get infections (such as high doses of steroids or immunosuppressant medicine)
they are pregnant
have a problem with spleen or the spleen has been removed (splenectomy)
they have Down's syndrome
having dialysis or have severe (stage 5) long-term kidney disease
classed as clinically extremely vulnerable, based on clinical judgement and an assessment of their needs
are 70 years old or older
have a lung condition that is not severe (such as asthma, COPD, emphysema or bronchitis)
have heart disease (such as heart failure)
have diabetes
have liver disease (such as hepatitis)
have a condition affecting the brain or nerves (such as Parkinson's disease, motor neurone disease, multiple sclerosis or cerebral palsy
they are very obese (for example a BMI of 40 or above).
   The Prognosis which is Determined

The process of the invention preferably determines prognosis for the severity of disease caused by coronavirus, and preferably by Covid-19. Therefore the process may determine whether the individual has a prognosis of severe disease or mild disease. The process may determine whether the individual has prognosis of severe disease that will lead: to the need for ICU treatment, a cytokine storm (cytokine release syndrome), hyperinflammation or sepsis.

### Tables Provided Herein

Tables 1, 2, 3 and 4 show specific markers which can be used to detect coronavirus infection, i.e. their presence or absence can be used in such a detection (i.e. they are ‘disseminating’ markers). Tables 1 and 2 show markers which are present in individuals that have the prognosis of severe disease. Tables 3 and 4 shows markers which are present in individuals that have prognosis of mild disease. Tables 2 and 4 are subsets of markers from Table 1 and 3 respectively. The process of the invention can be carried out using markers from any one of Tables 1 to 4 or by using markers from more than one Table, for example using markers from both Table 1 and Table 3.

Table 7 shows a further set of markers which can be used to detect prognosis for coronavirus infection. The process of the invention may be carried out using only the markers of Table 7, or these markers may be combined with other markers as disclosed herein.

Tables 8, 9, 10, 11, 12, 13 and 14 show further sets of markers which can be used to detect prognosis for coronavirus infection. The process of the invention may be carried out using only the markers of any of Table 8, 9, 10, 11, 12, 13 and 14 or these markers may be combined with other markers as disclosed herein.

Table 8 includes markers which are associated with the severe phenotype. Markers where there is a ‘1’ in the LS column are associated with severe phenotype in this table. These are preferred markers to be used in the invention.

Table 9 shows preferred markers which are all associated with severe phenotype.

Table 10 includes markers which are associated with mild phenotype. Markers where there is a ‘-1’ in the LS column are associated with mild phenotype. These are preferred markers to be used in the invention.

Table 11 shows preferred markers which are all associated with mild phenotype.

Tables 12 and 13 show markers which can be used to determine sepsis status. The results in Table 12 relate to “S_SS” and “qPCR_ICU”. These are 27 markers which are ICU prognosis markers but have also been found to be significant and sepsis-specific in the analysis of patients with sepsis vs severe sepsis. The results in Table 13 relate to “H_SS” and “qPCR_ICU”. These are 5 markers are ICU prognosis markers that also come as significant and severe-sepsis specific in the analysis of patients with severe sepsis when comparing to healthy.

Tables 14 and 15 show preferred severe (ICU) disease markers, the ‘1’ in the CCS column representing that phenotype.

Tables 16 and 17 show preferred mild disease markers, the ‘-1’ in the CCS column representing that phenotype.

The markers are defined using probe sequences (which detect a ligated product as defined herein). The first two sets of Start-End positions show probe positions, and the second two sets of Start-End positions show the relevant 4 kb region. The markers may be defined with reference to the Start-End positions which are provided as these will uniquely identify the marker in the same way a probe sequence does.

The following information is provided in the probe data tables:
RP—Rsum the Rank Product statistics evaluated per each chromosome interaction.
FC—Interaction frequency (positive or negative).
Pfp—estimated percentage of false positive predictions (pfp), both considering positive and negative chromosome interactions.
Pval—estimated pvalues per each CCSs being positive and negative.
Adj.P.value (FDR)—False discovery rate adjusted p.value.
Loop Detected—which state the loop is found in.

Simple permutation-based estimation is used to determine how likely a given RP value or better is observed in a random experiment. This has the following steps:
1. Generate p permutations of k rank lists of length n.
2. Calculate the rank products of the n CCS in the p permutations.
3. Count (c) how many times the rank products of the CCS in the permutations are smaller or equal to the observed rank product. Set c to this value.
4. Calculate the average expected value for the rank product by: Erp(g)=c/p.
5. Calculate the percentage of false positives as: pfp(g)=Erp(g)/rank (g) where rank(g) is the rank of CCS g in a list of all n CCSs sorted by increasing RP.

The rank product statistic ranks chromosome interactions according to intensities within each microarray and calculates the product of these ranks across multiple microarrays. This technique can identify chromosome interactions that are consistently detected among the most differential chromosome interactions in a number of replicated microarrays. Where the p-value is 0 this indicates that there is very little variation in the Rank Product of the CCS across the samples, this is a good example of the signal to noise and effect size of CCS. Where p value is 0 and pfp is 0 this means that permutated Rank Product doesn't differ from the actual observed Rank Product. These methods are described Breitling R and Herzyk P (2005) Rank-based methods as a non-parametric alternative of the t-test for the analysis of biological microarray data. J Bioinf Comp Biol 3, 1171-1189.

The FC indicates prevalence of marker in each comparison, 2 means twice over average test, 1.5 means 1.5 over the average test, etc., and so FC indicates the weight of a marker to phenotype/group. The FC value can be used to give an indication of how many markers are needed for a highly effective test. Individual markers are powerful indicators of prognosis, and typically 5 to 10 markers will give a highly effective test, though smaller numbers of markers will give a functional test for detection of coronavirus prognosis.

The probes are designed to be 30 bp away from the Taq1 site. In case of PCR, PCR primers are typically designed to detect ligated product but their locations from the Taq1 site vary. Probe locations:
Start 1—30 bases upstream of TaqI site on fragment 1
End 1—TaqI restriction site on fragment 1
Start 2—TaqI restriction site on fragment 2
End 2—30 bases downstream of TaqI site on fragment 2 4 kb Sequence Location:
Start 1—4000 bases upstream of TaqI site on fragment 1
End 1—TaqI restriction site on fragment 1
Start 2—TaqI restriction site on fragment 2
End 2—4000 bases downstream of TaqI site on fragment 2

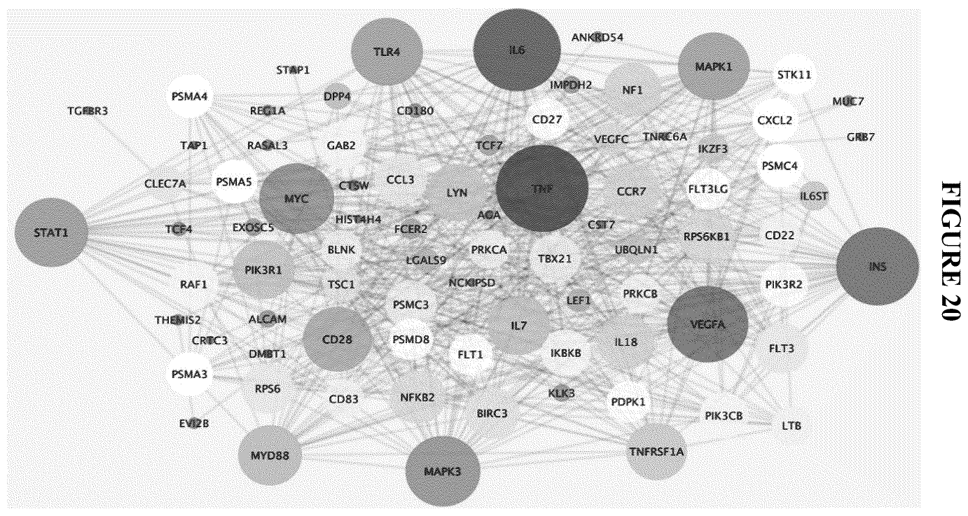

Table 5 relates to the network analysis shown in FIG. 20 and shows the String functional sub-networks represented in this network, with the names of the key genes in the sub-network associated with EpiSwitch markers.

Table 6 provides a list of therapeutic compounds which have sets of affected gene associated with them (see #genes column) and have individual genes from that sets (see following columns #matching genes) associated with EpiSwitch significant markers specific for severe disease outcome (all of them being immune associated genes). In the table:
the column with total number of genes shows genes affected by the therapeutic agent
the column with matched genes shows the number of affected genes matching EpiSwitch markers.

The high scores that were obtained confirm the non-random selection of the matching genes, i.e. affected by 3D genome architecture in severe disease patients.

Table 7 shows a set of preferred markers for use in the invention, as well as preferred probe and PCR primers. The specific prognosis the presence of each marker is associated with is shown in the first column: ‘Mild’ denotes the clinical manifestation of COVID disease (as opposed to asymptomatic conditions), where the patient may even be hospitalised, but responds to a standard of care and remains stable; ‘ICU’ denotes a severe condition when the hospitalized patient does not respond to a standard of care on the hospital ward and requires Intensive Care Unit (ICU) support. Examples of severe (ICU) are hyperinflammation and need of mechanical ventilation.

Table 8 shows a set of markers for use in the invention. Any marker may be used from this table. Preferred markers have a ‘1’ in the LS column are associated with severe phenotype in this table, and so all these markers represent a preferred subset of markers from which markers can be chosen for use in the invention.

Table 9 shows markers which are all associated with severe phenotype. The table also shows preferred primer and probe sequences. These primers and probes can be used in a qPCR format for detection of the relevant marker.

Table 10 shows a set of markers for use in the invention. Any marker may be used from this table. Preferred markers have a ‘-1’ in the LS column are associated with mild phenotype in this table, and so all these markers represent a preferred subset of markers from which markers can be chosen for use in the invention.

Table 11 shows markers which are all associated with mild phenotype. The table also shows preferred primer and probe sequences. These primers and probes can be used in a qPCR format for detection of the relevant marker.

Tables 14 and 16 show preferred probe and primer sequences which can optionally be used in a qPCR format. Tables 15 and 17 show preferred primer sequences which can optionally be used in a nested PCR format.

### Preferred Marker Sets

The invention relates to detecting prognosis for coronavirus infection by typing chromosome interaction markers, such as any of the specific markers disclosed herein, for example in Table 1 or 3, or preferred combinations of markers, or markers in defined specific regions disclosed herein. Markers present in genes and regions mentioned in the tables may be typed. Specific markers are defined herein by location or by probe and/or primer sequences. Therefore preferred markers are those which are represented by the probes and/or primer pairs disclosed in tables herein.

The invention relates to detecting prognosis for coronavirus infection by typing chromosome interaction markers, such as any of the specific markers disclosed Table 8, 9, 10, 11, 14, 15, 16 or 17 or preferred combinations of markers from any of these tables, or markers in defined specific regions disclosed in any of these tables.

The invention relates to further detecting sepsis status during coronavirus infection by typing chromosome interaction markers, such as any of the specific markers disclosed Table 12 or 13, or preferred combinations of markers from any of these tables.

Combinations of markers can be defined in different ways, such as:
by ranking by any parameter defined herein, or
by any ‘part’ of Table 1 or 3,
by reference to the ‘number’ of the marker which is listed in the left hand column of the Tables.

In a preferred aspect at least 10 markers are typed from the top 40 markers for any parameter mentioned in the Tables, such as FC.

In one aspect one or more markers are typed which:
(i) are present in any one of the regions listed in Table 1 or 3; and/or
(ii) corresponds to any one of the chromosome interactions represented by any probe shown in Table 1 or 3; and/or
(iii) is present in a 4,000 base region which comprises or which flanks (i) or (ii).

In a preferred aspect:
at least 5 chromosome interactions are typed from Table 1, and/or
at least 5 chromosome interactions are typed from Table 3.

In another preferred aspect at least 5 chromosome interactions are typed selected from:
the top 40 interactions in Table 1 defined using any parameter, and/or
the top 40 interactions in Table 3 defined using any parameter.

Combinations of markers can be defined by any ‘part’ of Table 8, 9, 10, 11, 12, 13, 14, 15, 16 or 17.

In a preferred aspect at least 10 markers are typed from the top 40 markers for any parameter mentioned in the Tables, such as FC.

In one aspect one or more markers are typed which:
(i) are present in any one of the regions listed in Table 8, 9, 10 or 11; and/or
(ii) corresponds to any one of the chromosome interactions represented by any probe shown in Table 8, 9, 10 or 11; and/or
(iii) is present in a 4,000 base region which comprises or which flanks (i) or (ii).

In a preferred aspect:
at least 5 chromosome interactions are typed from Table 9, and/or
at least 5 chromosome interactions are typed from Table 11.

In another preferred aspect at least 5 chromosome interactions are typed selected from:
the top 40 interactions in Table 8 defined using any parameter, and/or
the top 40 interactions in Table 10 defined using any parameter.

### Preferred Numbers of Markers to be Typed

Typing a very low number of the markers disclosed herein will result in an effective test due to the nature of regulation by chromosome interaction, including their network-like properties. The different numbers and combination of markers give rise to different performance properties. Further as will be appreciated the markers can be selected from Table 1 or 3 as a whole or from the parts of the Tables defined by a number and letter (for example ‘a2’). Similarly markers can be selected from the whole or parts of any of Tables 8, 9, 10, 11, 14, 15, 16 or 17. Markers can also be selected from the whole or parts of Tables 12 and 13.

In one aspect the process comprises typing at least 3, 5, 8, 10, 15, 20, 50, 100, 150, 200, 250 or 300 of the chromosome interactions represented by the probes in Table 1. In one embodiment at least 10 chromosome interactions represented by the probes in Table 1 are typed.

In one aspect the process comprises typing at least 3, 5, 8, 10, 15, 20, 25 or 30 of the chromosome interactions represented by the probes in Table 2. In one embodiment at least 10 chromosome interactions represented by the probes in Table 2 are typed.

In one aspect the process comprises typing at least 3, 5, 8, 10, 15, 20, 50, 80, 100, 150 or 200 of the chromosome interactions represented by the probes in Table 3.

In one aspect the process comprises typing at least 3, 5, 8 or 10 of the chromosome interactions represented by the probes in Table 4. In one embodiment at least 10 chromosome interactions represented by the probes in Table 4 are typed.

In one aspect at least 3, 5, 8, 10, 15 or 20 chromosome interactions are typed from the top 40 markers in Table 1 defined using any parameter and/or at least 3, 5, 8, 10, 15 or 20 chromosome interactions are typed from the top 40 markers in Table 3 defined using any parameter.

In one aspect 2, 3, 4, 5 or 6 markers are types from Table 7. All 6 of the markers of Table 7 may be typed. In another aspect all 6 of the markers from Table 7 are typed together with at least 3, 5, 8, 10, 15 or 20 chromosome interactions from Table 1. In another aspect all 6 of the markers of Table 7 are typed together with at least 3, 5, 8, 10, 15 or 20 markers from Table 3.

In one aspect the process comprises typing at least 3, 5, 8, 10, 15, 20, 50, 100, 150, 200, 250 or 300 of the chromosome interactions represented by the probes in Table 8. Preferably at least 10 chromosome interactions represented by the probes in Table 8 are typed.

In one aspect the process comprises typing at least 3, 5, 8, 10, 15, 20, 50, 100, or all of the chromosome interactions shown in Table 8 which have a ‘1’ in the LS column. Preferably at least 10 chromosome interactions shown Table 8 which have ‘1’ in the LS column are typed.

In one aspect the process comprising typing at least 3, 5, 8, 10, 15, 20, 30 or all of the chromosome interactions shown in Table 9. Preferably at least 10 chromosome interactions represented by the probes in Table 9 are typed.

In one aspect the process comprises typing at least 3, 5, 8, 10, 15, 20, 50, 100, 150, 200 or all of the chromosome interactions represented by the probes in Table 10. Preferably at least 10 chromosome interactions represented by the probes in Table 10 are typed.

In one aspect the process comprises typing at least 3, 5, 8, 10, 15, 20, 50, 80, or all of the chromosome interactions shown in Table 10 which have a ‘-1’ in the LS column. Preferably at least 10 chromosome interactions shown Table 10 which have ‘-1’ in the LS column are typed.

In one aspect the process comprises typing at least 3, 5, 8, 10 or all of the chromosome interactions shown in Table 11. Preferably at least 10 chromosome interactions represented by the probes in Table 11 are typed.

In one aspect the process comprises typing at least 3, 5, 8, 10, 20, 25 or all of the chromosome interactions shown in Table 12. Preferably at least 10 chromosome interactions represented by the probes in Table 12 are typed.

In one aspect the process comprises typing at least 2, 3 or 5 of the chromosome interactions shown in Table 13. Preferably at least 5 chromosome interactions represented by the probes in Table 13 are typed.

In one aspect the process comprises typing at least 3, 5, 8, 10, 15, 20, 50, 70 or all of the chromosome interactions represented by the probes in Table 14. Preferably at least 10 chromosome interactions represented by the probes in Table 14 are typed.

In one aspect the process comprises typing at least 3, 5, 8, 10, 15, 20, 50, 100, 120, or all of the chromosome interactions represented by the probes in Table 15. Preferably at least 10 chromosome interactions represented by the probes in Table 15 are typed.

In one aspect the process comprises typing at least 3, 5, 8, 10, 15, 20, 40, or all of the chromosome interactions represented by the probes in Table 16. Preferably at least 10 chromosome interactions represented by the probes in Table 16 are typed.

In one aspect the process comprises typing at least 3, 5, 8, 10, 15, 20, 50, 80 or all of the chromosome interactions represented by the probes in Table 17. Preferably at least 10 chromosome interactions represented by the probes in Table 17 are typed.

### Types of Chromosome Interaction

In one aspect the locus (including the gene and/or place where the chromosome interaction is detected) may comprise a CTCF binding site. This is any sequence capable of binding transcription repressor CTCF. That sequence may consist of or comprise the sequence CCCTC which may be present in 1, 2 or 3 copies at the locus. The CTCF binding site sequence may comprise the sequence CCGCGNGGNGGCAG (in IUPAC notation). The CTCF binding site may be within at least 100, 500, 1000 or 4000 bases of the chromosome interaction or within any of the chromosome regions shown Table 1.

When detection is performed using a probe, typically sequence from both regions of the probe (i.e. from both sites of the chromosome interaction) could be detected. In preferred aspects probes are used in the process which comprise or consist of the same or complementary sequence to a probe shown in any table. In some aspects probes are used which comprise sequence which is homologous to any of the probe sequences shown in the tables.

### The Approach Taken to Identify Markers and Panels of Markers

The invention described herein relates to chromosome conformation profile and 3D architecture as a regulatory modality in its own right, closely linked to the phenotype. The discovery of biomarkers was based on annotations through pattern recognition and screening on representative cohorts of clinical samples representing the differences in phenotypes. We annotated and screened significant parts of the genome, across coding and non-coding parts and over large sways of non-coding 5′ and 3′ of known genes for identification of statistically disseminating consistent conditional disseminating chromosome conformations, which for example anchor in the non-coding sites within (intronic) or outside of open reading frames.

In selection of the best markers we are driven by statistical data and p values for the marker leads. Selected and validated chromosome conformations within the signature are disseminating stratifying entities in their own right, irrespective of the expression profiles of the genes used in the reference. Further work may be done on relevant regulatory modalities, such as SNPs at the anchoring sites, changes in gene transcription profiles, changes at the level of H3K27ac.

We are taking the question of clinical phenotype differences and their stratification from the basis of fundamental biology and epigenetic controls over phenotype-including for example from the framework of network of regulation. As such, to assist stratification, one can capture changes in the network and it is preferably done through signatures of several biomarkers, for example through following a machine learning algorithm for marker reduction which includes evaluating the optimal number of markers to stratify the testing cohort with minimal noise. This may end with 3-20 markers.

Selection of markers for panels may be done by cross-validation statistical performance (and not for example by the functional relevance of the neighbouring genes, used for the reference name).

A panel of markers (with names of adjacent genes) is a product of clustered selection from the screening across significant parts of the genome, in non-biased way analysing statistical disseminating powers over 14,000-60,000 annotated EpiSwitch sites across significant parts of the genome. It should not be perceived as a tailored capture of a chromosome conformation on the gene of know functional value for the question of stratification. The total number of sites for chromosome interaction are 1.2 million, and so the potential number of combinations is 1.2 million to the power 1.2 million. The approach that we have followed nevertheless allows the identifying of the relevant chromosome interactions.

The specific markers that are provided by this application have passed selection, being statistically (significantly) associated with the condition or subgroup. This is what the data in the relevant table demonstrates. Each marker can be seen as representing an event of biological epigenetic as part of network deregulation that is manifested in the relevant condition. In practical terms it means that these markers are prevalent across groups of patients when compared to controls. On average, as an example, an individual marker may typically be present in 80% of patients tested and in 10% of controls tested.

Simple addition of all markers would not directly represent the network interrelationships between some of the deregulations. This is where the standard multivariate biomarker analysis GLMNET (R package) can be brought in. GLMNET package helps to identify interdependence between some of the markers, that reflect their joint role in achieving deregulations leading to disease phenotype. Modelling and then testing markers with highest GLMNET scores offers not only identify the minimal number of markers that accurately identifies the patient cohort, but also the minimal number that offers the least false positive results in the control group of patients, due to background statistical noise of low prevalence in the control group. Typically a group (combination) of selected markers (such as 3 to 10) offers the best balance between both sensitivity and specificity of detection, emerging in the context of multivariate analysis from individual properties of all the selected statistical significant markers for the condition.

The tables herein show the reference names for the array probes (60-mer) for array analysis that overlaps the juncture between the long range interaction sites, the chromosome number and the start and end of two chromosomal fragments that come into juxtaposition. The name of each marker listed in a table gives the chromosome position numbers of the two regions which are recognised by the relevant probe, providing an alternative way of defining the chromosome interaction in a unique way.

### Samples and Sample Treatment

The process of the invention will normally be carried out on a sample. The sample may be obtained at a defined time point, for example at any time point defined herein. The sample will normally contain DNA from the individual. It will normally contain cells. In one aspect a sample is obtained by minimally invasive means, and may for example be a blood sample. DNA may be extracted and cut up with a standard restriction enzyme. This can pre-determine which chromosome conformations are retained and will be detected with the EpiSwitch™ platforms. Due to the synchronisation of chromosome interactions between tissues and blood, including horizontal transfer, a blood sample can be used to detect the chromosome interactions in tissues, such as tissues relevant to disease.

### Preferred Aspects for Sample Preparation and Chromosome Interaction Detection

Methods of preparing samples and detecting chromosome conformations are described herein. Optimised (non-conventional) versions of these processes can be used, for example as described in this section.

Typically the sample will contain at least 2×10⁵ cells. The sample may contain up to 5×10⁵ cells. In one aspect, the sample will contain 2×10⁵ to 5.5×10⁵ cells.

Crosslinking of epigenetic chromosomal interactions present at the chromosomal locus is described herein. This may be performed before cell lysis takes place. Cell lysis may be performed for 3 to 7 minutes, such as 4 to 6 or about 5 minutes. In some aspects, cell lysis is performed for at least 5 minutes and for less than 10 minutes.

Digesting DNA with a restriction enzyme is described herein. Typically, DNA restriction is performed at about 55° C. to about 70° C., such as for about 65° C., for a period of about 10 to 30 minutes, such as about 20 minutes.

Preferably a frequent cutter restriction enzyme is used which results in fragments of ligated DNA with an average fragment size up to 4000 base pair. Optionally the restriction enzyme results in fragments of ligated DNA have an average fragment size of about 200 to 300 base pairs, such as about 256 base pairs. In one aspect, the typical fragment size is from 200 base pairs to 4,000 base pairs, such as 400 to 2,000 or 500 to 1,000 base pairs.

In one aspect of the EpiSwitch process a DNA precipitation step is not performed between the DNA restriction digest step and the DNA ligation step.

DNA ligation is described herein. Typically the DNA ligation is performed for 5 to 30 minutes, such as about 10 minutes.

The protein in the sample may be digested enzymatically, for example using a proteinase, optionally Proteinase K. The protein may be enzymatically digested for a period of about 30 minutes to 1 hour, for example for about 45 minutes. In one aspect after digestion of the protein, for example Proteinase K digestion, there is no cross-link reversal or phenol DNA extraction step.

In one aspect PCR detection is capable of detecting a single copy of the ligated nucleic acid, preferably with a binary read-out for presence/absence of the ligated nucleic acid.

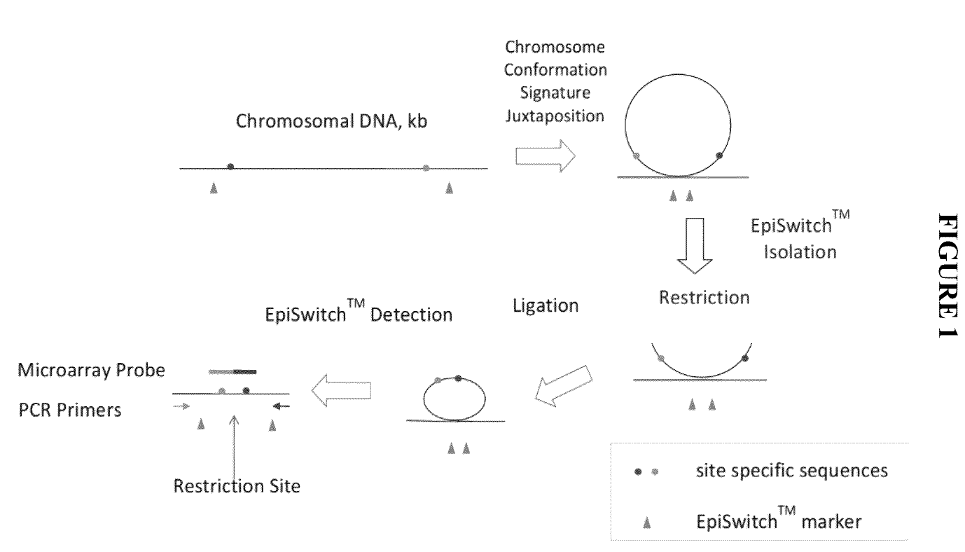

FIG. 1 shows a preferred process of detecting chromosome interactions.

### Processes and Uses of the Invention

The process of the invention can be described in different ways. It can be described as a process of making a ligated nucleic acid comprising (i) in vitro cross-linking of chromosome regions which have come together in a chromosome interaction; (ii) subjecting said cross-linked DNA to cutting or restriction digestion cleavage; and (iii) ligating said cross-linked cleaved DNA ends to form a ligated nucleic acid, wherein detection of the ligated nucleic acid may be used to determine the chromosome state at a locus, and wherein preferably:
the locus may be any of the loci or regions mentioned in Table 1, 3, 8 or 10 and/or
wherein the chromosomal interaction may be any of the chromosome interactions mentioned herein or corresponding to any of the probes disclosed in Table 1, 3, 8 or 10 and/or
wherein the ligated product may have or comprise (i) sequence which is the same as or homologous to any of the probe sequences disclosed in Table 1, 3, 8 or 10; or (ii) sequence which is complementary to (ii).

The process of the invention can be described as a process for detecting chromosome states which represent different subgroups in a population comprising determining whether a chromosome interaction is present or absent within a defined epigenetically active region of the genome, wherein preferably:
the subgroup is defined by prognosis for coronavirus infection, and/or
the chromosome state may be at any locus or region mentioned in Table 1 or 3; and/or
the chromosome interaction may be any of those mentioned in Table 1 or 3, or corresponding to any of the probes disclosed in those tables.

One or more of the markers of Table 7 may be used in these aspects of the invention, for example 3, 4, 5 or all of the markers of Table 7.

Further one or more of the markers of Table 8, 9, 10, 11, 12, 13, 14, 15, 16 or 17 may be used in these aspects of the invention, including specific numbers or combinations or markers from any of these tables as disclosed herein.

### Homologues

Homologues of polynucleotide/nucleic acid (e.g. DNA) sequences are referred to herein. Such homologues typically have at least 70% homology, preferably at least 80%, at least 85%, at least 90%, at least 95%, at least 97%, at least 98% or at least 99% homology, for example over a region of at least 10, 15, 20, 30, 100 or more contiguous nucleotides, or across the portion of the nucleic acid which is from the region of the chromosome involved in the chromosome interaction. The homology may be calculated on the basis of nucleotide identity (sometimes referred to as “hard homology”).

Therefore, in a particular aspect, homologues of polynucleotide/nucleic acid (e.g. DNA) sequences are referred to herein by reference to percentage sequence identity. Typically such homologues have at least 70% sequence identity, preferably at least 80%, at least 85%, at least 90%, at least 95%, at least 97%, at least 98% or at least 99% sequence identity, for example over a region of at least 10, 15, 20, 30, 100 or more contiguous nucleotides, or across the portion of the nucleic acid which is from the region of the chromosome involved in the chromosome interaction. The homologues may have at least 70% sequence identity, preferably at least 80%, at least 85%, at least 90%, at least 95%, at least 97%, at least 98% or at least 99% sequence identity across the entire probe, primer or primer pair.

For example the UWGCG Package provides the BESTFIT program which can be used to calculate homology and/or % sequence identity (for example used on its default settings) (Devereux et al (1984) Nucleic Acids Research 12, p 387-395). The PILEUP and BLAST algorithms can be used to calculate homology and/or % sequence identity and/or line up sequences (such as identifying equivalent or corresponding sequences (typically on their default settings)), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W5 T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P (N)), which provides an indication of the probability by which a match between two polynucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs by 1, 2, 3, 4 or more bases, such as less than 10, 15 or 20 bases (which may be substitutions, deletions or insertions of nucleotides). These changes may be measured across any of the regions mentioned above in relation to calculating homology and/or % percentage sequence identity.

Homology of a ‘pair of primers’ can be calculated, for example, by considering the two sequences as a single sequence (as if the two sequences are joined together) for the purpose of then comparing against the another primer pair which again is considered as a single sequence.

### EpiSwitch™ Technology

The EpiSwitch™ Technology also relates to the use of microarray EpiSwitch™ marker data in the detection of epigenetic chromosome conformation signatures specific for phenotypes. Aspects such as EpiSwitch™ which utilise ligated nucleic acids in the manner described herein have several advantages. They have a low level of stochastic noise, for example because the nucleic acid sequences from the first set of nucleic acids of the present invention either hybridise or fail to hybridise with the second set of nucleic acids. This provides a binary result permitting a relatively simple way to measure a complex mechanism at the epigenetic level. EpiSwitch™ technology also has fast processing time and low cost. In one aspect the processing time is 3 hours to 6 hours.

### Arrays

All nucleic acids disclosed herein may be bound to an array, and in one aspect there are at least 15,000, 45,000, 100,000 or 250,000 different nucleic acids bound to the array, which preferably represent at least 300, 900, 2000 or 5000 loci. In one aspect one, or more, or all of the different populations of nucleic acids are bound to more than one distinct region of the array, in effect repeated on the array allowing for error detection. The array may be based on an Agilent SurePrint G3 Custom CGH microarray platform. Detection of binding of first nucleic acids to the array may be performed by a dual colour system.

### The Threshold of Detection

The markers which are disclosed herein have been found to be ‘disseminating markers’ capable of determining coronavirus infection status or subgroup. In practical terms it means that these markers are prevalent across groups of patients when compared to controls (as is shown by the FC value, for example). On average, as an example, an individual marker may typically be present in 80% of patients tested and in 10% of controls tested. Thus in one aspect of the method an individual is deemed to be part of the relevant coronavirus prognosis subgroup if least 80% of the markers that are tested for that subgroup are present in the individual and/or if at least 80% of the markers that are tested which are related to the control are absent from the individual.

### Therapeutic Agents and Treatments

This section is relevant both to:
therapeutic agents which are given to individuals selected by the process of the invention, and
therapeutic agents which are selected based on the results of the process of the invention.

The invention provides therapeutic agents for use in preventing or treating coronavirus infection or related sub-condition in certain individuals, for example those identified by a process of the invention. This may comprise administering to an individual in need a therapeutically effective amount of the agent. The invention provides use of the agent in the manufacture of a medicament to prevent or treat a condition in certain individuals. The disease or condition may be coronavirus infection, any type of coronavirus infection sub-condition, such as severe disease, or a stage of coronavirus infection.

The formulation of the agent will depend upon the nature of the agent. The agent will be provided in the form of a pharmaceutical composition containing the agent and a pharmaceutically acceptable carrier or diluent. Suitable carriers and diluents include isotonic saline solutions, for example phosphate-buffered saline. Typical oral dosage compositions include tablets, capsules, liquid solutions and liquid suspensions. The agent may be formulated for parenteral, intravenous, intramuscular, subcutaneous, transdermal or oral administration.

The dose of an agent may be determined according to various parameters, especially according to the substance used; the age, weight and condition of the individual to be treated; the route of administration; and the required regimen. A physician will be able to determine the required route of administration and dosage for any particular agent. A suitable dose may however be from 0.1 to 100 mg/kg body weight such as 1 to 40 mg/kg body weight, for example, to be taken from 1 to 3 times daily.

The therapeutic agent may be any such agent disclosed herein, or may target any ‘target’ disclosed herein, including any protein or gene disclosed herein in any table. It is understood that any agent that is disclosed in a combination should be seen as also disclosed for administration individually.

Therapeutic agents and treatments which can be used in the invention include the following:

### Antivirals

Remdesivir-reduces lung virus and lung damage.
Lopinavir, ritonavir or a lopinavir/ritonavir combination-reduces lung virus and lung damage.
Umifenovir-inhibits viral entry into target cells and stimulate immune response.
   Immune modulators
Dexamethasone—for dampening down the body's immune system.
Convalescent plasma—contains high levels of polyclonal, pathogen specific antibodies. These antibodies may confer passive immunity to recipients.

The individual may be treated with any therapeutic agent selected from Table 6, and preferably Abatacept, Afelimomab, Angiotensin II, dexamethasone, Imatinib, immunoglobulin, Infliximab, Nintedanib, Rituximab or Ulixertinib.

Individuals identified as having prognosis for severe disease:
can be treated for hyperinflammation
can be given specialized treatment under close observation
can be treated with agents to reduce viral load
can be given immunomodulators
can be given a vaccine
can be given prophylactic treatment
can be provided with quarantine isolation
can be given therapeutic agents which prevent or treat sepsis.

### Further Therapy Embodiments

The invention provides therapy of individual to prevent or treat any prognosis mentioned herein (including prognosis to severe coronavirus disease), such as any type of individual mentioned herein (including defined by risk factors, disease, susceptibility or any other characteristic mentioned herein). The individual may have been identified as being susceptible to severe coronavirus disease. Preferably the invention provides therapy to prevent or treat severe coronavirus disease, optionally in an individual who has been identified as being susceptible of to such disease. The individual may be given any therapy mentioned herein, including the any of the agents listed in Table 6.

The invention provides a therapeutic agent selected from any of the agents shown in Table 6 for use in a method of treatment of severe coronavirus disease, said method comprising:
identifying whether an individual is susceptible to severe coronavirus disease by the process of the invention (i.e. by typing chromosome interactions in the individual), and
administering to any individual identified as being susceptible said agent.

The invention provides a method of treatment comprising identifying whether an individual is susceptible to severe coronavirus disease by the typing method of the invention and administering to any individual identified as being susceptible any agent listed in Table 6.

Preferred agents from Table 6 include Abatacept, Afelimomab, Angiotensin II, dexamethasone, Imatinib, immunoglobulin, Infliximab, Nintedanib, Rituximab and Ulixertinib.

The invention provides an agent which:
prevents or treats coronavirus infection, and/or
prevents or treats a detrimental immune response;
   for use in treating an individual that has been identified as being susceptible to severe disease as a result of coronavirus infection according to the method of the invention.

### Personalised Therapy

The invention provides different types of personalised treatment. In one aspect an individual is given or not given therapy based on the results of the method of the invention, i.e. based on the detecting of the presence or absence one or more specific chromosome interactions. The method of the invention is in this sense typically allowing selection of a therapy based on the individual's prognosis or responsiveness to the therapy.

In one aspect the invention provides a method of treatment of an individual comprising:
(i) detecting the presence or absence of one or more chromosome interactions represented by the probes shown in Table 1 or 3,
(ii) administering to the individual a therapeutic agent which is selected based on the presence or absence of said chromosome interactions in the individual.

In one aspect a therapeutic agent is selected which targets a particular gene or gene product (for example the expressed protein) where the gene is one in which the chromosome interaction is associated with or present within.

In the personalised therapy aspects of the invention any specific chromosome interaction can be typed which is mentioned herein, for example in any table. Any number or combination of interactions disclosed herein can be typed. One or more of the markers of Table 7 may be used in these aspects of the invention, for example 3, 4, 5 or all of the markers of Table 7. Further one or more of the markers of Table 8, 9, 10, 11, 12, 13, 14, 15, 16 or 17 may be used in these aspects of the invention, including specific numbers or combinations or markers from any of these tables as disclosed herein.

### Screening for Therapeutic Agents

The invention provides a method of screening candidate agents (for example compounds) to determine whether they can be used for therapy of any condition mentioned herein, including susceptibility to severe coronavirus disease, preferably to severe Covid-19 disease.

In one aspect the invention provides a method of determining whether a candidate agent is therapeutic for severe coronavirus disease comprising determining whether the candidate is able to alter one or more chromosome interactions disclosed herein, including the numbers and combinations of chromosome interaction disclosed herein, for example as represented by the probes shown in Table 1 or 3. This method may determine whether the candidate agent is able create or destroy such interaction(s).

In one aspect the invention provides a method for determining whether a candidate agent can be used to prevent or treat severe coronavirus disease comprising;
(i) contacting the candidate agent with one or more chromosomes,
(ii) determining whether the candidate agent is able to create or destroy one or more chromosome interactions represented by the probes shown in Table 1 or 3 on said chromosomes,
to thereby determine whether the candidate agent can be used to prevent or treat severe coronavirus disease.

One or more of the markers of Table 7 may be used in these aspects of the invention, for example 3, 4, 5 or all of the markers of Table 7. Further one or more of the markers of Table 8, 9, 10, 11, 12, 13, 14, 15, 16 or 17 may be used in these aspects of the invention, including specific numbers or combinations or markers from any of these tables as disclosed herein.

### Properties of Nucleic Acids of the Invention

The invention relates to certain nucleic acids, such as the ligated nucleic acids which are described herein as being used or generated in the process of the invention. These may be the same as, or have any of the properties of, the first and second nucleic acids mentioned herein. The nucleic acids of the invention typically comprise two portions each comprising sequence from one of the two regions of the chromosome which come together in the chromosome interaction. Typically each portion is at least 8, 10, 15, 20, 30 or 40 nucleotides in length, for example 10 to 40 nucleotides in length. Preferred nucleic acids comprise sequence from any of the genes mentioned in any of the tables. Typically preferred nucleic acids comprise the specific probe sequences mentioned in Table 1 or 3; or fragments and/or homologues of such sequences.

Preferably the nucleic acids are DNA. It is understood that where a specific sequence is provided the invention may use the complementary sequence as required in the particular aspect. Preferably the nucleic acids are DNA. It is understood that where a specific sequence is provided the invention may use the complementary sequence as required in the particular aspect.

The primers shown in Table 1 or 3 may also be used in the invention as mentioned herein. In one aspect primers are used which comprise any of: the sequences shown in Table 1 or 3; or fragments and/or homologues of any sequence shown in Table 1 or 3.

One or more of the probes or primer pairs of Table 7 may be used in these aspects of the invention, for example 3, 4, 5 or all of probes or primer pairs of Table 7. Further one or more of the probes or primers of any of Table 8, 9, 10, 11, 12, 13, 14, 15, 16 or 17 may be used in these aspects of the invention.

### Screening to Identify Relevant Chromosome Interactions

In one aspect one or more of the chromosome interactions which are typed have been identified by a process of determining which chromosomal interactions are relevant to a chromosome state corresponding to a coronavirus infection subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to the subgroup.

The second set of nucleic acid sequences has the function of being a set of index sequences, and is essentially a set of nucleic acid sequences which are suitable for identifying subgroup specific sequence. They can represents the ‘background’ chromosomal interactions and might be selected in some way or be unselected. They are in general a subset of all possible chromosomal interactions.

The second set of nucleic acids may be derived by any suitable process. They can be derived computationally or they may be based on chromosome interaction in individuals. They typically represent a larger population group than the first set of nucleic acids. In one particular aspect, the second set of nucleic acids represents all possible epigenetic chromosomal interactions in a specific set of genes. In another particular aspect, the second set of nucleic acids represents a large proportion of all possible epigenetic chromosomal interactions present in a population described herein. In one particular aspect, the second set of nucleic acids represents at least 50% or at least 80% of epigenetic chromosomal interactions in at least 20, 50, 100 or 500 genes, for example in 20 to 100 or 50 to 500 genes.

The second set of nucleic acids typically represents at least 100 possible epigenetic chromosome interactions which modify, regulate or in any way mediate a phenotype in population. The second set of nucleic acids may represent chromosome interactions that affect a disease state (typically relevant to diagnosis or prognosis) in a species. The second set of nucleic acids typically comprises sequences representing epigenetic interactions both relevant and not relevant to a prognosis subgroup.

In one particular aspect the second set of nucleic acids derive at least partially from naturally occurring sequences in a population, and are typically obtained by in silico processes. Said nucleic acids may further comprise single or multiple mutations in comparison to a corresponding portion of nucleic acids present in the naturally occurring nucleic acids. Mutations include deletions, substitutions and/or additions of one or more nucleotide base pairs. In one particular aspect, the second set of nucleic acids may comprise sequence representing a homologue and/or orthologue with at least 70% sequence identity to the corresponding portion of nucleic acids present in the naturally occurring species. In another particular aspect, at least 80% sequence identity or at least 90% sequence identity to the corresponding portion of nucleic acids present in the naturally occurring species is provided.

### Properties of the Second Set of Nucleic Acids

In one particular aspect, there are at least 100 different nucleic acid sequences in the second set of nucleic acids, preferably at least 1000, 2000 or 5000 different nucleic acids sequences, with up to 100,000, 1,000,000 or 10,000,000 different nucleic acid sequences. A typical number would be 100 to 1,000,000, such as 1,000 to 100,000 different nucleic acids sequences. All or at least 90% or at least 50% or these would correspond to different chromosomal interactions.

In one particular aspect, the second set of nucleic acids represent chromosome interactions in at least 20 different loci or genes, preferably at least 40 different loci or genes, and more preferably at least 100, at least 500, at least 1000 or at least 5000 different loci or genes, such as 100 to 10,000 different loci or genes. The lengths of the second set of nucleic acids are suitable for them to specifically hybridise according to Watson Crick base pairing to the first set of nucleic acids to allow identification of chromosome interactions specific to subgroups. Typically the second set of nucleic acids will comprise two portions corresponding in sequence to the two chromosome regions which come together in the chromosome interaction. The second set of nucleic acids typically comprise nucleic acid sequences which are at least 10, preferably 20, and preferably still 30 bases (nucleotides) in length. In another aspect, the nucleic acid sequences may be at the most 500, preferably at most 100, and preferably still at most 50 base pairs in length. In a preferred aspect, the second set of nucleic acids comprises nucleic acid sequences of between 17 and 25 base pairs. In one aspect at least 100, 80% or 50% of the second set of nucleic acid sequences have lengths as described above. Preferably the different nucleic acids do not have any overlapping sequences, for example at least 100%, 90%, 80% or 50% of the nucleic acids do not have the same sequence over at least 5 contiguous nucleotides.

Given that the second set of nucleic acids acts as an ‘index’ then the same set of second nucleic acids may be used with different sets of first nucleic acids which represent subgroups for different characteristics, i.e. the second set of nucleic acids may represent a ‘universal’ collection of nucleic acids which can be used to identify chromosome interactions relevant to different characteristics.

### The First Set of Nucleic Acids (Screening for Relevant Chromosome Interactions)

The first set of nucleic acids are typically from subgroups relevant to coronavirus infection. The first nucleic acids may have any of the characteristics and properties of the second set of nucleic acids mentioned herein. The first set of nucleic acids is normally derived from samples from the individuals which have undergone treatment and processing as described herein, particularly the EpiSwitch™ cross-linking and cleaving steps. Typically the first set of nucleic acids represents all or at least 80% or 50% of the chromosome interactions present in the samples taken from the individuals.

Typically, the first set of nucleic acids represents a smaller population of chromosome interactions across the loci or genes represented by the second set of nucleic acids in comparison to the chromosome interactions represented by second set of nucleic acids, i.e. the second set of nucleic acids is representing a background or index set of interactions in a defined set of loci or genes.

### Library of Nucleic Acids

Any of the types of nucleic acid populations mentioned herein may be present in the form of a library comprising at least 200, at least 500, at least 1000, at least 5000 or at least 10000 different nucleic acids of that type, such as ‘first’ or ‘second’ nucleic acids. Such a library may be in the form of being bound to an array. The library may comprise some or all of the probes or primer pairs shown in any table disclosed herein. The library may comprise all of the probe sequence from any of the tables disclosed herein.

### Hybridisation

The invention typically requires a means for allowing wholly or partially complementary nucleic acid sequences to hybridise, for example in the method of the invention or between the first set of nucleic acids and the second set of nucleic acids to hybridise. In one aspect all of the first set of nucleic acids is contacted with all of the second set of nucleic acids in a single assay, i.e. in a single hybridisation step. However any suitable assay can be used.

### Labelled Nucleic Acids and Pattern of Hybridisation

The nucleic acids mentioned herein may be labelled, preferably using an independent label such as a fluorophore (fluorescent molecule) or radioactive label which assists detection of successful hybridisation. Certain labels can be detected under UV light. The pattern of hybridisation, for example on an array described herein, represents differences in epigenetic chromosome interactions between the two subgroups, and thus provides a process of comparing epigenetic chromosome interactions and determination of which epigenetic chromosome interactions are specific to a subgroup in the population of the present invention.

The term ‘pattern of hybridisation’ broadly covers the presence and absence of hybridisation, for example between the first and second set of nucleic acids, i.e. which specific nucleic acids from the first set hybridise to which specific nucleic acids from the second set, and so it not limited to any particular assay or technique, or the need to have a surface or array on which a ‘pattern’ can be detected.

### Forms of the Substance Mentioned Herein

Any of the substances, such as nucleic acids or therapeutic agents, mentioned herein may be in purified or isolated form. They may be in a form which is different from that found in nature, for example they may be present in combination with other substance with which they do not occur in nature. The nucleic acids (including portions of sequences defined herein) may have sequences which are different to those found in nature, for example having at least 1, 2, 3, 4 or more nucleotide changes in the sequence as described in the section on homology. The nucleic acids may have heterologous sequence at the 5′ or 3′ end. The nucleic acids may be chemically different from those found in nature, for example they may be modified in some way, but preferably are still capable of Watson-Crick base pairing. Where appropriate the nucleic acids will be provided in double stranded or single stranded form. The invention provides all of the specific nucleic acid sequences mentioned herein in single or double stranded form, and thus includes the complementary strand to any sequence which is disclosed.

The invention provides a kit for carrying out any process of the invention, including detection of a chromosomal interaction relating to prognosis. Such a kit can include a specific binding agent capable of detecting the relevant chromosomal interaction, such as agents capable of detecting a ligated nucleic acid generated by processes of the invention. Preferred agents present in the kit include probes capable of hybridising to the ligated nucleic acid or primer pairs, for example as described herein, capable of amplifying the ligated nucleic acid in a PCR reaction.

The invention provides a device that is capable of detecting the relevant chromosome interactions. The device preferably comprises any specific binding agents, probe or primer pair capable of detecting the chromosome interaction, such as any such agent, probe or primer pair described herein.

### Detection Process

In one aspect quantitative detection of the ligated sequence which is relevant to a chromosome interaction is carried out using a probe which is detectable upon activation during a PCR reaction, wherein said ligated sequence comprises sequences from two chromosome regions that come together in an epigenetic chromosome interaction, wherein said process comprises contacting the ligated sequence with the probe during a PCR reaction, and detecting the extent of activation of the probe, and wherein said probe binds the ligation site. The process typically allows particular interactions to be detected in a MIQE compliant manner using a dual labelled fluorescent hydrolysis probe.

The probe is generally labelled with a detectable label which has an inactive and active state, so that it is only detected when activated. The extent of activation will be related to the extent of template (ligation product) present in the PCR reaction. Detection may be carried out during all or some of the PCR, for example for at least 50% or 80% of the cycles of the PCR.

The probe can comprise a fluorophore covalently attached to one end of the oligonucleotide, and a quencher attached to the other end of the nucleotide, so that the fluorescence of the fluorophore is quenched by the quencher. In one aspect the fluorophore is attached to the 5′end of the oligonucleotide, and the quencher is covalently attached to the 3′ end of the oligonucleotide.

Fluorophores that can be used in the process of the invention include FAM, TET, JOE, Yakima Yellow, HEX, Cyanine3, ATTO 550, TAMRA, ROX, Texas Red, Cyanine 3.5, LC610, LC 640, ATTO 647N, Cyanine 5, Cyanine 5.5 and ATTO 680. Quenchers that can be used with the appropriate fluorophore include TAM, BHQ1, DAB, Eclip, BHQ2 and BBQ650, optionally wherein said fluorophore is selected from HEX, Texas Red and FAM. Preferred combinations of fluorophore and quencher include FAM with BHQ1 and Texas Red with BHQ2.

Use of the Probe in a qPCR Assay

Hydrolysis probes of the invention are typically temperature gradient optimised with concentration matched negative controls. Preferably single-step PCR reactions are optimized. More preferably a standard curve is calculated. An advantage of using a specific probe that binds across the junction of the ligated sequence is that specificity for the ligated sequence can be achieved without using a nested PCR approach. The processes described herein allow accurate and precise quantification of low copy number targets. The target ligated sequence can be purified, for example gel-purified, prior to temperature gradient optimization. The target ligated sequence can be sequenced. Preferably PCR reactions are performed using about 10 ng, or 5 to 15 ng, or 10 to 20 ng, or 10 to 50 ng, or 10 to 200 ng template DNA. Forward and reverse primers are designed such that one primer binds to the sequence of one of the chromosome regions represented in the ligated DNA sequence, and the other primer binds to other chromosome region represented in the ligated DNA sequence, for example, by being complementary to the sequence.

Detection of the ligated nucleic acid may use any probe and/or primer pair disclosed herein in any table. In one aspect a qPCR system is used which used the probes and primer pairs disclosed in Table 7, for homologues of such probes and primer pairs. Preferably the probes shown in the ‘Modifications Seq’ column of Table 7 are used with the types of quencher and reporter shown.

### Choice of Ligated DNA Target

The invention includes selecting primers and a probe for use in a PCR process as defined herein comprising selecting primers based on their ability to bind and amplify the ligated sequence and selecting the probe sequence based properties of the target sequence to which it will bind, in particular the curvature of the target sequence.

Probes are typically designed/chosen to bind to ligated sequences which are juxtaposed restriction fragments spanning the restriction site. In one aspect of the invention, the predicted curvature of possible ligated sequences relevant to a particular chromosome interaction is calculated, for example using a specific algorithm referenced herein. The curvature can be expressed as degrees per helical turn, e.g. 10.5° per helical turn. Ligated sequences are selected for targeting where the ligated sequence has a curvature propensity peak score of at least 5° per helical turn, typically at least 10°, 15° or 20° per helical turn, for example 5° to 20° per helical turn. Preferably the curvature propensity score per helical turn is calculated for at least 20, 50, 100, 200 or 400 bases, such as for 20 to 400 bases upstream and/or downstream of the ligation site. Thus in one aspect the target sequence in the ligated product has any of these levels of curvature. Target sequences can also be chosen based on lowest thermodynamic structure free energy.

### Particular Aspects

In one aspect only intrachromosomal interactions are typed/detected, and no extrachromosomal interactions (between different chromosomes) are typed/detected.

In particular aspects certain chromosome interactions are not typed, for example any specific interaction mentioned herein (for example as defined by any probe or primer pair mentioned herein). In some aspects chromosome interactions are not typed in any of the genes relevant to chromosome interactions mentioned herein.

The data provided herein shows that the markers are ‘disseminating’ ones able to differentiate cases and non-cases for the relevant disease situation, for example prognosis. Therefore when carrying out the invention the skilled person will be able to determine by detection of the interactions which subgroup the individual is in. In one embodiment a threshold value of detection of at least 70% of the tested markers in the form they are associated with the relevant disease situation (either by absence or presence) may be used to determine whether the individual is in the relevant subgroup.

### Screening Process

The invention provides a process of determining which chromosomal interactions are relevant to a chromosome state corresponding to an prognosis subgroup of the population, comprising contacting a first set of nucleic acids from subgroups with different states of the chromosome with a second set of index nucleic acids, and allowing complementary sequences to hybridise, wherein the nucleic acids in the first and second sets of nucleic acids represent a ligated product comprising sequences from both the chromosome regions that have come together in chromosomal interactions, and wherein the pattern of hybridisation between the first and second set of nucleic acids allows a determination of which chromosomal interactions are specific to an prognosis subgroup. The subgroup may be any of the specific subgroups defined herein, for example with reference to particular conditions or therapies.

### Disclosure in Publications and Priority Applications

The contents of all publications mentioned herein are incorporated by reference into the present specification and may be used to further define the features relevant to the invention. The contents of all priority applications are incorporated into the present specification and may be used to define the features relevant to the invention.

### Techniques Used to Identify the Specific Relevant Chromosome Interactions

The EpiSwitch™ platform technology detects epigenetic regulatory signatures of regulatory changes between normal and abnormal conditions at loci. The EpiSwitch™ platform identifies and monitors the fundamental epigenetic level of gene regulation associated with regulatory high order structures of human chromosomes also known as chromosome conformation signatures. Chromosome signatures are a distinct primary step in a cascade of gene deregulation. They are high order biomarkers with a unique set of advantages against biomarker platforms that utilize late epigenetic and gene expression biomarkers, such as DNA methylation and RNA profiling.

### EpiSwitch™ Array Assay

The custom EpiSwitch™ array-screening platforms come in 4 densities of, 15K, 45K, 100K, and 250K unique chromosome conformations, each chimeric fragment is repeated on the arrays 4 times, making the effective densities 60K, 180K, 400K and 1 million respectively.

### Custom Designed EpiSwitch™ Arrays

The 15K EpiSwitch™ array can screen the whole genome including around 300 loci interrogated with the EpiSwitch™ Biomarker discovery technology. The EpiSwitch™ array is built on the Agilent SurePrint G3 Custom CGH microarray platform; this technology offers 4 densities, 60K, 180K, 400K and 1 Million probes. The density per array is reduced to 15K, 45K, 100K and 250K as each EpiSwitch™ probe is presented as a quadruplicate, thus allowing for statistical evaluation of the reproducibility. The average number of potential EpiSwitch™ markers interrogated per genetic loci is 50, as such the numbers of loci that can be investigated are 300, 900, 2000, and 5000.

### EpiSwitch™ Custom Array Pipeline

The EpiSwitch™ array is a dual colour system with one set of samples, after EpiSwitch™ library generation, labelled in Cy5 and the other of sample (controls) to be compared/analyzed labelled in Cy3. The arrays are scanned using the Agilent SureScan Scanner and the resultant features extracted using the Agilent Feature Extraction software. The data is then processed using the EpiSwitch™ array processing scripts in R. The arrays are processed using standard dual colour packages in Bioconductor in R: Limma*. The normalisation of the arrays is done using the normalisedWithinArrays function in Limma* and this is done to the on chip Agilent positive controls and EpiSwitch™ positive controls. The data is filtered based on the Agilent Flag calls, the Agilent control probes are removed and the technical replicate probes are averaged, in order for them to be analysed using Limma*. The probes are modelled based on their difference between the 2 scenarios being compared and then corrected by using False Discovery Rate. Probes with Coefficient of Variation (CV)<=30% that are <=−1.1 or =>1.1 and pass the p<=0.1 FDR p-value are used for further screening. To reduce the probe set further Multiple Factor Analysis is performed using the FactorMineR package in R. * Note: LIMMA is Linear Models and Empirical Bayes Processes for Assessing Differential Expression in Microarray Experiments. Limma is an R package for the analysis of gene expression data arising from microarray or RNA-Seq.

The pool of probes is initially selected based on adjusted p-value, FC and CV<30% (arbitrary cut off point) parameters for final picking. Further analyses and the final list are drawn based only on the first two parameters (adj. p-value; FC).

### Statistical Pipeline

EpiSwitch™ screening arrays are processed using the EpiSwitch™ Analytical Package in R in order to select high value EpiSwitch™ markers for translation on to the EpiSwitch™ PCR platform.

### Step 1

Probes are selected based on their corrected p-value (False Discovery Rate, FDR), which is the product of a modified linear regression model. Probes below p-value <=0.1 are selected and then further reduced by their Epigenetic ratio (ER), probes ER have to be <=−1.1 or =>1.1 in order to be selected for further analysis. The last filter is a coefficient of variation (CV), probes have to be below <=0.3.

### Step 2

The top 40 markers from the statistical lists are selected based on their ER for selection as markers for PCR translation. The top 20 markers with the highest negative ER load and the top 20 markers with the highest positive ER load form the list.

### Step 3

The resultant markers from step 1, the statistically significant probes form the bases of enrichment analysis using hypergeometric enrichment (HE). This analysis enables marker reduction from the significant probe list, and along with the markers from step 2 forms the list of probes translated on to the EpiSwitch™ PCR platform.

The statistical probes are processed by HE to determine which genetic locations have an enrichment of statistically significant probes, indicating which genetic locations are hubs of epigenetic difference.

The most significant enriched loci based on a corrected p-value are selected for probe list generation. Genetic locations below p-value of 0.3 or 0.2 are selected. The statistical probes mapping to these genetic locations, with the markers from step 2, form the high value markers for EpiSwitch™ PCR translation.

### Array Design and Processing

### Array Design

Genetic loci are processed using the SII software (currently v3.2) to:
Pull out the sequence of the genome at these specific genetic loci (gene sequence with 50 kb upstream and 20 kb downstream)
Define the probability that a sequence within this region is involved in CCs
Cut the sequence using a specific RE
Determine which restriction fragments are likely to interact in a certain orientation
Rank the likelihood of different CCs interacting together.
Determine array size and therefore number of probe positions available (x)
Pull out x/4 interactions.
For each interaction define sequence of 30 bp to restriction site from part 1 and 30 bp to restriction site of part 2. Check those regions are not repeats, if so exclude and take next interaction down on the list. Join both 30 bp to define probe.
Create list of x/4 probes plus defined control probes and replicate 4 times to create list to be created on array
Upload list of probes onto Agilent Sure design website for custom CGH array.
Use probe group to design Agilent custom CGH array.

### Array Processing

Process samples using EpiSwitch™ Standard Operating Procedure (SOP) for template production.
Clean up with ethanol precipitation by array processing laboratory.
Process samples as per Agilent SureTag complete DNA labelling kit-Agilent Oligonucleotide Array-based CGH for Genomic DNA Analysis Enzymatic labelling for Blood, Cells or Tissues
Scan using Agilent C Scanner using Agilent feature extraction software.

EpiSwitch™ biomarker signatures demonstrate high robustness, sensitivity and specificity in the stratification of complex disease phenotypes. This technology takes advantage of the latest breakthroughs in the science of epigenetics, monitoring and evaluation of chromosome conformation signatures as a highly informative class of epigenetic biomarkers. Current research methods deployed in academic environment require from 3 to 7 days for biochemical processing of cellular material in order to detect CCSs. Those procedures have limited sensitivity, and reproducibility; and furthermore, do not have the benefit of the targeted insight provided by the EpiSwitch™ Analytical Package at the design stage.

### EpiSwitch™ Array in Silico Marker Identification

CCS sites across the genome are directly evaluated by the EpiSwitch™ Array on clinical samples from testing cohorts for identification of all relevant stratifying lead biomarkers. The EpiSwitch™ Array platform is used for marker identification due to its high-throughput capacity, and its ability to screen large numbers of loci rapidly. The array used was the Agilent custom-CGH array, which allows markers identified through the in silico software to be interrogated.

### EpiSwitch™ PCR

Potential markers identified by EpiSwitch™ Array are then validated either by EpiSwitch™ PCR or DNA sequencers (i.e. Roche 454, Nanopore MinION, etc.). The top PCR markers which are statistically significant and display the best reproducibility are selected for further reduction into the final EpiSwitch™ Signature Set, and validated on an independent cohort of samples. EpiSwitch™ PCR can be performed by a trained technician following a standardised operating procedure protocol established. All protocols and manufacture of reagents are performed under ISO 13485 and 9001 accreditation to ensure the quality of the work and the ability to transfer the protocols. EpiSwitch™ PCR and EpiSwitch™ Array biomarker platforms are compatible with analysis of both whole blood and cell lines. The tests are sensitive enough to detect abnormalities in very low copy numbers using small volumes of blood.

### Use of a Classifier

The method of the invention may include analysis of the chromosome interactions identified in the individual, for example using a classifier, which may increase performance, such as sensitivity or specificity. The classifier is typically one that has been ‘trained’ on samples from the population and such training may assist the classifier to detect any prognosis (including susceptibility) mentioned herein.

The invention is illustrated by the following:

### EXAMPLE

Identification of Chromosome Interaction Markers Using the EpiSwitch™ Platform which Represent Changes in Chromosome Conformations Due to Infection with the Sars-Covid-2 Virus

Chromosome interaction markers were identified based on being the top immunogenetic markers and also on ‘pure stats’, including having other biological links to important clinical observations such as hypercalcaemia (markers around Ca homeostasis disruption, calmodulin, calcineurin, etc.).

The prognostic stratification was based on blood collections shortly after a Covid positive test, either in asymptomatic individuals, or in people just admitted to hospital. Stratification was based on outcomes of mild Covid disease, which manifests itself in hospitalized patients, who stay of the wards and respond to treatments, and severe Covid disease which we associate with patients being transferred to ICU (intensive care unit). Severe patients do not respond to treatments available on the hospital words (extra oxygen, anti-inflammatoires, etc.) and deteriorate to ICU emergency support. The stratifications provide prognosis of immune health and hyperinflammmation in individuals, when exposed to Covid infection.

The marker data is based on 80 patients from a mixture of cohorts from UK, USA and Peru, representing asymptomatic, mild and ICU severe cases. 42 samples came from Lima, Peru, collected at the time of Lima being the world hotspot for the highest number of complications and fatalities from Covid-19 (Peru has highest per capita mortality—107 per 100,000, for comparison in the US—71 per 100, 000; in Brazil—76 per 100,000). There were 11 samples from the UK and 19 from the US.

### Experimental Work

Whole blood samples were taken from patients hospitalised with confirmed COVID-19 disease (symptoms of SARS-CoV-2 infection) and split into two definitions: severe outcomes and mild outcomes. The severe outcome definition includes patient that were admitted to hospital and required more aggressive intervention in ICU care, or where this intervention would have been provided except for comorbidities. Patients that were admitted to hospital that received only oxygen treatment and or less invasive interventions on the ward (not admitted to ICU) are defined as mild.

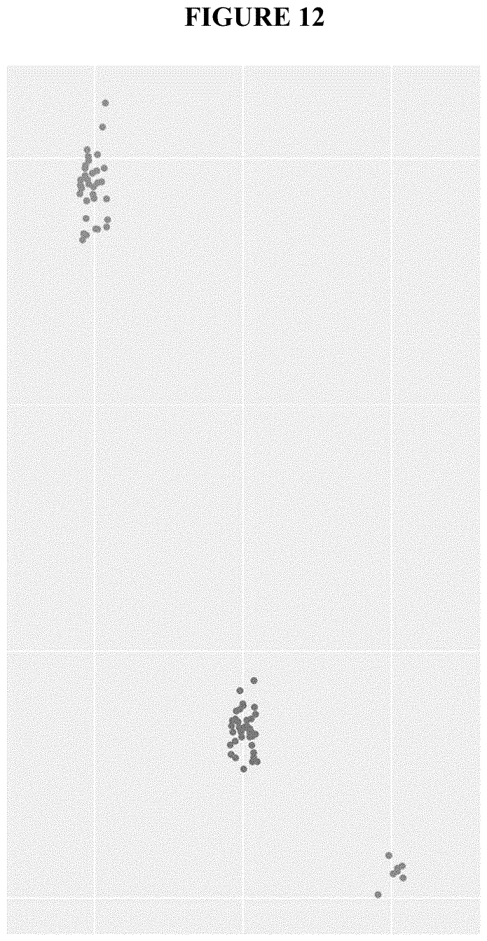

The EpiSwitch™ Microarray platform was initially utilised to interrogate patient samples with known clinical outcomes to gain biological insight and identify a group of potential markers that can delimit between the severe and mild patient samples. The aim was to identify the most statistically significant and biologically relevant markers. This study is done on the whole genome array, with over 900,000 selected anchor sites interrogated for each of the 80 patients presented in LDA analysis in FIG. 12, all reduced to a linear score.

The top EpiSwitch™ markers were subject to primer design to identify interactions that can be successfully interrogated with Nested PCR (nPCR) in the laboratory. The top markers from the list of the successfully designed interactions were selected and used to translate the microarray markers into nPCR markers though screening of the top 150 markers on the complete sample cohort.

The work had three primary aims: to investigate Covid-19 in whole blood samples, identify the top microarray interactions and translate them into nPCR, and provide proof of concept for a Covid-19 severity whole-blood based EpiSwitch™ nPCR product.

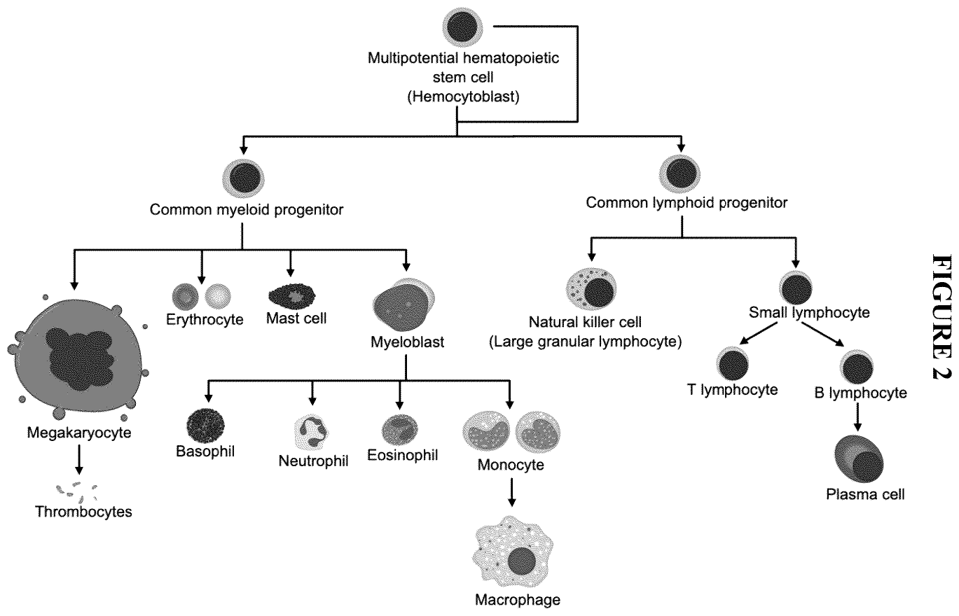

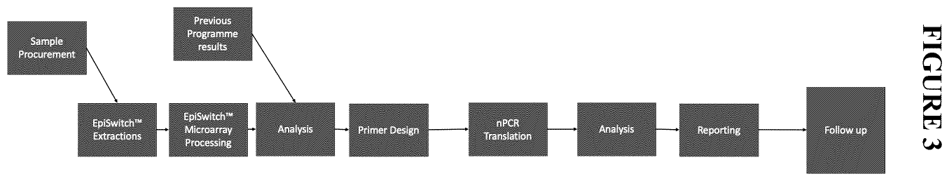

Whole blood and PBMC often contain different cell populations, with a large proportion of the granulocytes (basophil, neutrophil and eosinophil) found in whole blood being lost during the density gradient processing to purify the PBMC fraction from whole blood (see FIG. 2). This must be borne in mind when interpreting results. The study design is shown in FIG. 3.

42 patients were enrolled into the collection for this part of the work. A venous blood sample was taken from each subject. A volume of 5 ml or more of whole blood was be taken for EpiSwitch™ biochemical processing.

### Inclusion Criteria

Subject must be enrolled within 72 hours of presentation to the hospital

Subjects were recruited into the following two groups:
severe cases: 24 patients in ICU and/or mechanically ventilated
mild COVID-19:18 patients that are not in ICU, and also not mechanically ventilated, but just hospitalised (they can be receiving supplemental oxygen via nasal cannula).

Whole blood samples were collected in EDTA K3 blood collection tubes. The clinical annotations accompanying the samples consisted of:
demographics
Travel History
Symptoms
Date of Symptom Onset
Pre-Existing Medical Conditions
SARS-CoV-2 RT-PCR Result
Date of Specimen Collection
Other SOC Respiratory Diagnostic Result (if available)
Date of Hospitalisation
Date of Admitted to ICU (if applicable)
Information in regard to use of mechanical ventilation and duration of use
Information in regard to supplemental oxygen and duration of use

After blood sampling the EDTA tubes were inverted to mix the sample with the EDTA coating on the surface of the tubes to avoid clots. Sample were stored at −20° C. or lower, ideally within 60 minutes of collection.

### EpiSwitch Library Preparation

The 42 procured samples were processed using the EpiSwitch™ extraction protocols. The prepared libraries were quantified with Picogreen and Nanoquant (dsDNA and total nucleic acid measurements respectively). Each of the prepared EpiSwitch™ libraries were quality controlled using a standard OBD nPCR positive control assay before being stored at −80° C. until used in the subsequent steps.

After the required volume of EpiSwitch library was generated for the Microarray processing the extractions were continued to generate the required volume of library for the nPCR translation.

### Microarray Processing

The EpiSwitch™ Whole Genome Medium Density Protein Coding focussed Array was utilised using single channel analysis. The array consists of 973,335 EpiSwitch™ interactions probes and 2500 EpiSwitch™ control probes. The design focuses around protein coding and long non-coding RNA loci in the genome (GRCh38).

Each of the 42 EpiSwitch™ libraries were processed and labelled for a single channel microarray using Cy3 dye only. Each processed library was hybridised to a separate microarray. The in-line control cocktail consisting of four external DNA fragments was used to provide quality control and quantification.

### Microarray Analysis

The EpiSwitch Microarray data was analysed to identify differentially detected interactions between the two different disease phenotypes conditions. After the top markers were identified in the statistical and biological analysis the nPCR primer combinations were designed for each putative marker. Only markers that have primer combinations designed were translated to a nPCR assay. The default parameters of the Metagenome primer design were initially used to design optimal primer combinations for the markers followed by raising or lowering parameter thresholds as required.

### EpiSwitch™ Discovery Nested PCR Translation

For the top 150 markers identified from the Microarray and primer design the physical primers were ordered to allow for screening of nPCR assays that can differentiate between the severe and mild disease phenotypes. The entire 42 sample cohort was used for the nPCR translation. Each patient library was normalised to a 1 ng/ul dsDNA concentration and a serial dilution consisting of 1×, 1:2×, and 1:4× generated for assay screening.

The end point nPCR products were run on the LabChip GX Touch High throughput capillary electrophoresis machine using the 5K Chip and reagent option. Appropriate controls, including a negative human genomic control were used for each assay to ensure the products detected are actual chromosome conformation capture products and not non-specific binding of high copy number genomic DNA. The NTC was used to detect any cross contamination of reagents.

### EpiSwitch™ Discovery Nested PCR Analysis

The EpiSwitch Nested PCR platform data output was analysed with multiple statistical techniques including but not limited to Fishers Exact test, GLMNET (logistic Regression), and Bayesian logistic regression. For development of EpiSwitch™ classifiers the following statistical analysis were used:
XGBoost. A gradient boosted decision tree algorithm. An ensemble of weak decision tree models is generated and combined to produce one strong classification model.
Logistic Principal Component Analysis, optimised to use binary data.
GLMNET. Generalised linear model fitted via penalised maximum likelihood.

### Results and Treatment Implications

The markers identified in the above work are shown in Tables 1 to 4 and the Figures shows additional data and results. Separate sets of nPCR and qPCR markers were developed in parallel as shown in the tables. Selection of qPCR markers (when compared to nPCR) from the array relied on an additional filter for high abundance. In the tables, if a marker by its genomic position overlaps with immune-genetic loci (genetic marker for immune controls) it is marked as “immune” and if it coincides with cardiovascular genetic markers-“cardio”.

A prognostic test based on the identified markers is a measure of immune-health and immune competence under the exposure to Covid-19 infection. The high risk group (with a prognosis for severe disease) are likely to develop hyperinflammation and show poor response to standard of care treatment, alerting physicians to the necessity of specialized treatment under close observation: reduction of viral load and introduction of immunomodulators. Furthermore, in the context of vaccine use and distribution, the test identifies high risk groups who should be subject to vaccination. At the same time, among the general working population it could help identify high risk groups for further prophylactic treatment, protection and quarantine isolation, whilst the low risk group could be supported in returning to work.

### Identification of Gene Pathways

Chromosome interaction markers correspond to a regulatory network. Our analysis looked at how the markers corresponded to biological pathways. We obtained statistical scores showing relative overlap of significant EpiSwitch 3D genomic markers with genetic loci representing particular pathways. The higher the score the more genes from that pathway are co-localized with the sites dysregulated in the genome architecture. This showed the biological relevance of pathways, as distinct to individual genes, at the level of 3D genomics showing. The identified pathways show how the markers relate to immune health (systemic immune competence) beyond a Covid-19 model of disease.

The top pathways affected at genetic locations by 3D dysregulation include:
Innate Immune System
Toll-like Receptor Signaling Pathway
ERK Signaling
TGF-beta Signaling Pathway (WikiPathways)
Cytokine Signaling in Immune System
TCR Signaling (REACTOME)
Th17 Cell Differentiation
Class I MHC Mediated Antigen Processing and Presentation
Apoptosis Pathway
Rheumatoid Arthritis
Immune Response NFAT in Immune Response
ICos-ICosL Pathway in T-Helper Cell
Signaling By GPCR
Allograft Rejection
IL12 Signaling Mediated By STAT4
Immune Response Function of MEF2 in T Lymphocytes
CXCR4-mediated Signaling Events
Apoptotic Pathways in Synovial Fibroblasts
GPCR Pathway.

FIG. 20 shows a standard STRING network analysis that was carried out and Table 5 shows the names of the key genes in the network associated with EpiSwitch markers.

### Identified Therapies

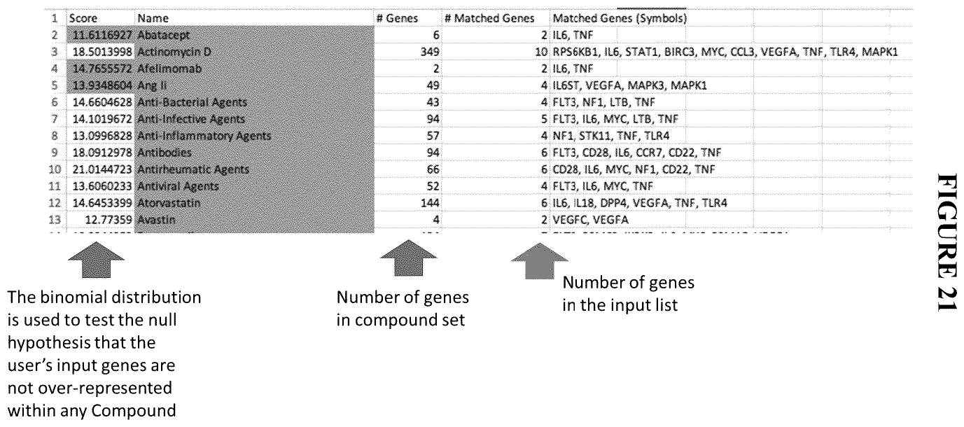

Therapeutic compounds were identified using the sets of affected genes and gene sets associated with EpiSwitch significant markers specific for the severe disease group. Table 6 shows the list of compounds and FIG. 21 describes how to interpret this table.

### Conclusions

The present work clearly indicates that severe/ICU (critical care unit) is a separate phenotype from mild or asymptomatic patients when it comes to 3D genomic profiling with EpiSwitch markers (i.e. chromosome interaction markers). Moreover, this is seen prognostically in advance. The markers that were identified are strongly localized around genes responsible for immunity. De facto this means that through the 3D genomics of the immune-genetic part of the genome there is immune-health predisposition in individuals. This predisposition means that if certain individuals are exposed to Covid-19 infection they will deteriorate due to the nature of their immune response and will not respond to standard treatment (hence they are moved to ICU). They undergo hyperinflammation, often targeting specific organs.

### Further Work Relating to the Markers of Table 7

Marker Selection from Array

The data is loaded and normalized using R Limma package, first the Agilent control probes are removed, next step is to exclude any probes that are above the saturation point for the system 65,525, this is due to the range of detection by the scanners. The next two steps relate to normalization of the data, first there is a background correction, then a normalization between the arrays is performed using a quantile approach, this standardizes the probes on the arrays and between arrays, both these steps minimize non-biological variation.

Directly after normalization the positive and spike in controls are removed prior to statistical analysis. Two statistical procedures are adopted for statistical analysis, linear modelling using the LIMMA package and Rank Product analysis using BigRankProduct package. The linear analysis is a parametric approach and Rank Product a non-parametric approach. In order for the probes to be statistically significant they have to be below <=0.05 for the adjusted p-value. The statistic lists from both approaches are compared and the union of the two is restricted further by two steps. The next filter on the combined statistical list is abundance, probes have to be either −1.2<= or =>1.2 in the abundance scale. This list is then further filtered for translation to qPCR by considering the normalized intensity of the samples, all samples have to be above =>1000 in intensity units or =>9.66 in log 2 scale. Once these filters have been applied the data is ranked by the probes abundance value (descending for positive integers, and ascending for negative integers), the top 120-200 markers from the positive and negative selection, form the list of markers used in translation to the qPCR platform.

### Final C19 Models

The final binary outcome COVID19 model has been built using WHO classification where severity is determined if the patients have been ventilated (see below).

ASTERIX Severity Groups for Cohort Partitioning

| | | SARS- | | | Severe Resp Failure | Critical Respiratory | |
|---|---|---|---|---|---|---|---|
| | | COV-2 | COVID | Admitted to | (>Step 6i.e. Max | Failure requiring | |
| | | PCR+ | Pneumonia | Hospital | 02%260% or CPAP) | ventilation | Dead |
| 1 | Mild | YES | Not required but | NO | NO | NO | NO |
| | | | report if YES | | | | |
| 2 | Moderate | YES | Not required but | YES | NO | NO | NO |
| | | | report if YES | | | | |
| 3 | Severe | YES | Not required but | YES | YES | NO | NO |
| | | | report if YES | | | | |
| 4 | Very | YES | Not required but | YES | Not required but | YES | NO |
| | Severe | | report if YES | | report if YES | | |
| 5 | Fatal | YES | Not required but | Not required but | Not required but | Not required but | YES |
| | | | report if YES | report if YES | report if YES | report if YES | |

| | | |
|---|---|---|
| ↓ | ↓ | ↓ |
| This corresponds to | This corresponds to | This corresponds to |
| Clinical Progression | Clinical Progression | Clinical Progression |
| Model 1 in the | Model 2 in the | Model 3 in the |
| protocol/SAP | protocol/SAP | protocol/SAP |

The at-risk patients (very severe, Yes to ventilation) are ones who need ventilation, and the ones not requiring ventilation form the other set in this classifier, average-risk (No to ventilation). Two boosting classifiers form the bases of the CST model, XGBoost (Extreme Gradient Boosting) and CatBoost (gradient boosting on decision trees), the probabilities from these models are combined and the call of high-risk are patients who achieve a combined probability score=>0.75, the average-risk patients are ones whose combined probability score is below <0.75.

### Final Combined Model Stats

The first confusion table is the training and 2 test sets combined.

Statistics (n=116) for prediction of High Risk (Critical/Severe (ICU))
Accuracy: 92%
Sensitivity: 96%
Specificity: 86%
PPV: 92%
NPV: 93%

See below:

### Confusion Matrix and Statistics

Reference

| Prediction | No | Yes |
|---|---|---|
| No | 38 | 3 |
| Yes | 6 | 69 |

Accuracy: 0.9224
95% CI: (0.8578, 0.9639)
No Information Rate: 0.6207
P-Value [Acc >NIR]: 9.843e-14
Kappa: 0.833
Mcnemar's Test P-Value: 0.505
Sensitivity: 0.9583
Specificity: 0.8636
Pos Pred Value: 0.9200
Neg Pred Value: 0.9268
Prevalence: 0.6207
Detection Rate: 0.5948
Detection Prevalence: 0.6466
Balanced Accuracy: 0.9110
‘Positive’ Class: Yes

The second is the 2 test sets combined: 28 ventilated patients (mixture of ICU (18) and not (10)) and patients who died (11 ICU, 8 non ICU, who were not ventilated). These 2 sets were not used in the model building. This is shown below:

| | |
|---|---|
| > | cm.m_3<−confusionMatrix(as.factor(c$Ventilation), |

as.factor(c$call),positive=“Yes”)

| | |
|---|---|
| > | cm.m_3 |

combined
Confusion Matrix and Statistics

| | Reference | | |
|---|---|---|---|
| Prediction | | No | Yes |
| | No | 38 | 3 |
| | Yes | 6 | 69 |

| | |
|---|---|
| Accuracy | : 0.9224 |
| 95% CI | : (0.8578, 0.9639) |

| | |
|---|---|
| No Information Rate | : 0.6207 |
| P-Value [Acc > NIR] | : 9.843e−14 |

| | |
|---|---|
| Kappa | : 0.833 |

| Mcnemar's Test P-Value | | : 0.505 |
|---|---|---|
| | Sensitivity | : 0.9583 |
| | Specificity | : 0.8636 |

| | |
|---|---|
| Pos Pred Value | : 0.9200 |
| Neg Pred Value | : 0.9268 |

| | |
|---|---|
| Prevalence | : 0.6207 |

| | |
|---|---|
| Detection Rate | : 0.5948 |
| Detection Prevalence | : 0.6466 |
| Balanced Accuracy | : 0.9110 |
| ‘Positive’ Class | : Yes |

test sets
Confusion Matrix and Statistics

| | Reference | | |
|---|---|---|---|
| Prediction | | No | Yes |
| | No | 5 | 3 |
| | Yes | 6 | 33 |

| | |
|---|---|
| Accuracy | : 0.8085 |
| 95% CI | : (0.6674, 0.9085) |

| | |
|---|---|
| No Information Rate | : 0.766 |
| P-Value [Acc > NIR] | : 0.311 |

| | |
|---|---|
| Kappa | : 0.41 |

| Mcnemar's Test P-Value | | : 0.505 |
|---|---|---|
| | Sensitivity | : 0.9167 |
| | Specificity | : 0.4545 |

| | |
|---|---|
| Pos Pred Value | : 0.8462 |
| Neg Pred Value | : 0.6250 |

| | |
|---|---|
| Prevalence | : 0.7660 |

| | |
|---|---|
| Detection Rate | : 0.7021 |
| Detection Prevalence | : 0.8298 |
| Balanced Accuracy | : 0.6856 |
| ‘Positive’ Class | : Yes |

| | |
|---|---|
| > | c %>% filter(Ventilation==“Yes” & In._ICU==“No”) %>% count( ) |
| | n |
| 1 | 16 |

>

### Further Conclusions

In terms of efficiency of testing a qPCR format has many advantages, with a highly effective design of the paired primers and probes providing high efficacy of detection.

Further technical advantages are gained by using a number of markers together in a test. Systemic epigenetic readouts based on EpiSwitch markers capture the functional set up of biological network regulation at cellular levels reflecting the underlying specific pathological phenotype. Stratification models may be based on groups of biomarkers that reflect regulatory network inter-relationships and synchronization between individual contributing factors. This can be combined with machine learning models and a decision can be taken on ‘optimised modelling’, including how many markers to use in a test.

### Sepsis Work

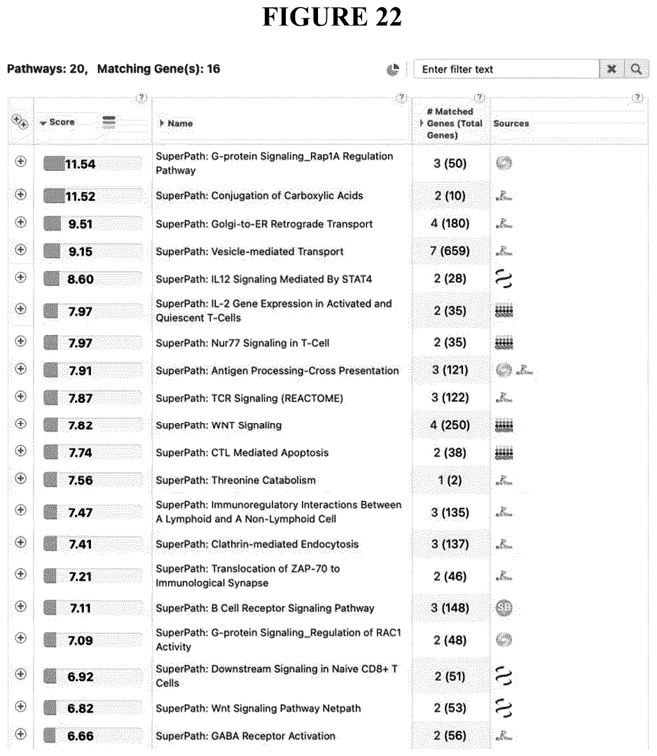

The markers were further analysed to identify those that correlated with sepsis phenotypes. The results are provided in Tables 12 and 13 and FIG. 22. It must be noted that PSMAS is a gene that has already been identified in the sepsis field as well as in Covid studies as a possible therapeutic target. The present work for the first time directly shows PSMAS locus involvement in the same dysregulation network at chromosome conformation level in both Covid and sepsis conditions, and provides markers for monitoring sepsis and determining prognosis of sepsis-like outcomes in coronavirus patients.

Lengthy table referenced here
US20260002208A1-20260101-T00001
Please refer to the end of the specification for access instructions.

Lengthy table referenced here
US20260002208A1-20260101-T00002
Please refer to the end of the specification for access instructions.

Lengthy table referenced here
US20260002208A1-20260101-T00003
Please refer to the end of the specification for access instructions.

Lengthy table referenced here
US20260002208A1-20260101-T00004
Please refer to the end of the specification for access instructions.

Lengthy table referenced here
US20260002208A1-20260101-T00005
Please refer to the end of the specification for access instructions.

Lengthy table referenced here
US20260002208A1-20260101-T00006
Please refer to the end of the specification for access instructions.

Lengthy table referenced here
US20260002208A1-20260101-T00007
Please refer to the end of the specification for access instructions.

Lengthy table referenced here
US20260002208A1-20260101-T00008
Please refer to the end of the specification for access instructions.

Lengthy table referenced here
US20260002208A1-20260101-T00009
Please refer to the end of the specification for access instructions.

Lengthy table referenced here
US20260002208A1-20260101-T00010
Please refer to the end of the specification for access instructions.

Lengthy table referenced here
US20260002208A1-20260101-T00011
Please refer to the end of the specification for access instructions.

Lengthy table referenced here
US20260002208A1-20260101-T00012
Please refer to the end of the specification for access instructions.

Lengthy table referenced here
US20260002208A1-20260101-T00013
Please refer to the end of the specification for access instructions.

Lengthy table referenced here
US20260002208A1-20260101-T00014
Please refer to the end of the specification for access instructions.

Lengthy table referenced here
US20260002208A1-20260101-T00015
Please refer to the end of the specification for access instructions.

Lengthy table referenced here
US20260002208A1-20260101-T00016
Please refer to the end of the specification for access instructions.

Lengthy table referenced here
US20260002208A1-20260101-T00017
Please refer to the end of the specification for access instructions.

Lengthy table referenced here
US20260002208A1-20260101-T00018
Please refer to the end of the specification for access instructions.

Lengthy table referenced here
US20260002208A1-20260101-T00019
Please refer to the end of the specification for access instructions.

Lengthy table referenced here
US20260002208A1-20260101-T00020
Please refer to the end of the specification for access instructions.

Lengthy table referenced here
US20260002208A1-20260101-T00021
Please refer to the end of the specification for access instructions.

Lengthy table referenced here
US20260002208A1-20260101-T00022
Please refer to the end of the specification for access instructions.

Lengthy table referenced here
US20260002208A1-20260101-T00023
Please refer to the end of the specification for access instructions.

Lengthy table referenced here
US20260002208A1-20260101-T00024
Please refer to the end of the specification for access instructions.

Lengthy table referenced here
US20260002208A1-20260101-T00025
Please refer to the end of the specification for access instructions.

Lengthy table referenced here
US20260002208A1-20260101-T00026
Please refer to the end of the specification for access instructions.

Lengthy table referenced here
US20260002208A1-20260101-T00027
Please refer to the end of the specification for access instructions.

Lengthy table referenced here
US20260002208A1-20260101-T00028
Please refer to the end of the specification for access instructions.

Lengthy table referenced here
US20260002208A1-20260101-T00029
Please refer to the end of the specification for access instructions.

Lengthy table referenced here
US20260002208A1-20260101-T00030
Please refer to the end of the specification for access instructions.

Lengthy table referenced here
US20260002208A1-20260101-T00031
Please refer to the end of the specification for access instructions.

Lengthy table referenced here
US20260002208A1-20260101-T00032
Please refer to the end of the specification for access instructions.

Lengthy table referenced here
US20260002208A1-20260101-T00033
Please refer to the end of the specification for access instructions.

Lengthy table referenced here
US20260002208A1-20260101-T00034
Please refer to the end of the specification for access instructions.

Lengthy table referenced here
US20260002208A1-20260101-T00035
Please refer to the end of the specification for access instructions.

Lengthy table referenced here
US20260002208A1-20260101-T00036
Please refer to the end of the specification for access instructions.

Lengthy table referenced here
US20260002208A1-20260101-T00037
Please refer to the end of the specification for access instructions.

Lengthy table referenced here
US20260002208A1-20260101-T00038
Please refer to the end of the specification for access instructions.

Lengthy table referenced here
US20260002208A1-20260101-T00039
Please refer to the end of the specification for access instructions.

Lengthy table referenced here
US20260002208A1-20260101-T00040
Please refer to the end of the specification for access instructions.

Lengthy table referenced here
US20260002208A1-20260101-T00041
Please refer to the end of the specification for access instructions.

Lengthy table referenced here
US20260002208A1-20260101-T00042
Please refer to the end of the specification for access instructions.

Lengthy table referenced here
US20260002208A1-20260101-T00043
Please refer to the end of the specification for access instructions.

Lengthy table referenced here
US20260002208A1-20260101-T00044
Please refer to the end of the specification for access instructions.

Lengthy table referenced here
US20260002208A1-20260101-T00045
Please refer to the end of the specification for access instructions.

Lengthy table referenced here
US20260002208A1-20260101-T00046
Please refer to the end of the specification for access instructions.

Lengthy table referenced here
US20260002208A1-20260101-T00047
Please refer to the end of the specification for access instructions.

Lengthy table referenced here
US20260002208A1-20260101-T00048
Please refer to the end of the specification for access instructions.

Lengthy table referenced here
US20260002208A1-20260101-T00049
Please refer to the end of the specification for access instructions.

Lengthy table referenced here
US20260002208A1-20260101-T00050
Please refer to the end of the specification for access instructions.

Lengthy table referenced here
US20260002208A1-20260101-T00051
Please refer to the end of the specification for access instructions.

Lengthy table referenced here
US20260002208A1-20260101-T00052
Please refer to the end of the specification for access instructions.

Lengthy table referenced here
US20260002208A1-20260101-T00053
Please refer to the end of the specification for access instructions.

Lengthy table referenced here
US20260002208A1-20260101-T00054
Please refer to the end of the specification for access instructions.

Lengthy table referenced here
US20260002208A1-20260101-T00055
Please refer to the end of the specification for access instructions.

Lengthy table referenced here
US20260002208A1-20260101-T00056
Please refer to the end of the specification for access instructions.

Lengthy table referenced here
US20260002208A1-20260101-T00057
Please refer to the end of the specification for access instructions.

Lengthy table referenced here
US20260002208A1-20260101-T00058
Please refer to the end of the specification for access instructions.

Lengthy table referenced here
US20260002208A1-20260101-T00059
Please refer to the end of the specification for access instructions.

Lengthy table referenced here
US20260002208A1-20260101-T00060
Please refer to the end of the specification for access instructions.

Lengthy table referenced here
US20260002208A1-20260101-T00061
Please refer to the end of the specification for access instructions.

Lengthy table referenced here
US20260002208A1-20260101-T00062
Please refer to the end of the specification for access instructions.

Lengthy table referenced here
US20260002208A1-20260101-T00063
Please refer to the end of the specification for access instructions.

Lengthy table referenced here
US20260002208A1-20260101-T00064
Please refer to the end of the specification for access instructions.

Lengthy table referenced here
US20260002208A1-20260101-T00065
Please refer to the end of the specification for access instructions.

Lengthy table referenced here
US20260002208A1-20260101-T00066
Please refer to the end of the specification for access instructions.

Lengthy table referenced here
US20260002208A1-20260101-T00067
Please refer to the end of the specification for access instructions.

Lengthy table referenced here
US20260002208A1-20260101-T00068
Please refer to the end of the specification for access instructions.

Lengthy table referenced here
US20260002208A1-20260101-T00069
Please refer to the end of the specification for access instructions.

Lengthy table referenced here
US20260002208A1-20260101-T00070
Please refer to the end of the specification for access instructions.

Lengthy table referenced here
US20260002208A1-20260101-T00071
Please refer to the end of the specification for access instructions.

Lengthy table referenced here
US20260002208A1-20260101-T00072
Please refer to the end of the specification for access instructions.

Lengthy table referenced here
US20260002208A1-20260101-T00073
Please refer to the end of the specification for access instructions.

Lengthy table referenced here
US20260002208A1-20260101-T00074
Please refer to the end of the specification for access instructions.

Lengthy table referenced here
US20260002208A1-20260101-T00075
Please refer to the end of the specification for access instructions.

Lengthy table referenced here
US20260002208A1-20260101-T00076
Please refer to the end of the specification for access instructions.

Lengthy table referenced here
US20260002208A1-20260101-T00077
Please refer to the end of the specification for access instructions.

Lengthy table referenced here
US20260002208A1-20260101-T00078
Please refer to the end of the specification for access instructions.

Lengthy table referenced here
US20260002208A1-20260101-T00079
Please refer to the end of the specification for access instructions.

Lengthy table referenced here
US20260002208A1-20260101-T00080
Please refer to the end of the specification for access instructions.

Lengthy table referenced here
US20260002208A1-20260101-T00081
Please refer to the end of the specification for access instructions.

Lengthy table referenced here
US20260002208A1-20260101-T00082
Please refer to the end of the specification for access instructions.

Lengthy table referenced here
US20260002208A1-20260101-T00083
Please refer to the end of the specification for access instructions.

Lengthy table referenced here
US20260002208A1-20260101-T00084
Please refer to the end of the specification for access instructions.

Lengthy table referenced here
US20260002208A1-20260101-T00085
Please refer to the end of the specification for access instructions.

Lengthy table referenced here
US20260002208A1-20260101-T00086
Please refer to the end of the specification for access instructions.

Lengthy table referenced here
US20260002208A1-20260101-T00087
Please refer to the end of the specification for access instructions.

Lengthy table referenced here
US20260002208A1-20260101-T00088
Please refer to the end of the specification for access instructions.

Lengthy table referenced here
US20260002208A1-20260101-T00089
Please refer to the end of the specification for access instructions.

Lengthy table referenced here
US20260002208A1-20260101-T00090
Please refer to the end of the specification for access instructions.

Lengthy table referenced here
US20260002208A1-20260101-T00091
Please refer to the end of the specification for access instructions.

Lengthy table referenced here
US20260002208A1-20260101-T00092
Please refer to the end of the specification for access instructions.

Lengthy table referenced here
US20260002208A1-20260101-T00093
Please refer to the end of the specification for access instructions.

Lengthy table referenced here
US20260002208A1-20260101-T00094
Please refer to the end of the specification for access instructions.

Lengthy table referenced here
US20260002208A1-20260101-T00095
Please refer to the end of the specification for access instructions.

Lengthy table referenced here
US20260002208A1-20260101-T00096
Please refer to the end of the specification for access instructions.

Lengthy table referenced here
US20260002208A1-20260101-T00097
Please refer to the end of the specification for access instructions.

Lengthy table referenced here
US20260002208A1-20260101-T00098
Please refer to the end of the specification for access instructions.

Lengthy table referenced here
US20260002208A1-20260101-T00099
Please refer to the end of the specification for access instructions.

Lengthy table referenced here
US20260002208A1-20260101-T00100
Please refer to the end of the specification for access instructions.

Lengthy table referenced here
US20260002208A1-20260101-T00101
Please refer to the end of the specification for access instructions.

Lengthy table referenced here
US20260002208A1-20260101-T00102
Please refer to the end of the specification for access instructions.

Lengthy table referenced here
US20260002208A1-20260101-T00103
Please refer to the end of the specification for access instructions.

Lengthy table referenced here
US20260002208A1-20260101-T00104
Please refer to the end of the specification for access instructions.

Lengthy table referenced here
US20260002208A1-20260101-T00105
Please refer to the end of the specification for access instructions.

Lengthy table referenced here
US20260002208A1-20260101-T00106
Please refer to the end of the specification for access instructions.

Lengthy table referenced here
US20260002208A1-20260101-T00107
Please refer to the end of the specification for access instructions.

Lengthy table referenced here
US20260002208A1-20260101-T00108
Please refer to the end of the specification for access instructions.

Lengthy table referenced here
US20260002208A1-20260101-T00109
Please refer to the end of the specification for access instructions.

Lengthy table referenced here
US20260002208A1-20260101-T00110
Please refer to the end of the specification for access instructions.

Lengthy table referenced here
US20260002208A1-20260101-T00111
Please refer to the end of the specification for access instructions.

Lengthy table referenced here
US20260002208A1-20260101-T00112
Please refer to the end of the specification for access instructions.

Lengthy table referenced here
US20260002208A1-20260101-T00113
Please refer to the end of the specification for access instructions.

Lengthy table referenced here
US20260002208A1-20260101-T00114
Please refer to the end of the specification for access instructions.

Lengthy table referenced here
US20260002208A1-20260101-T00115
Please refer to the end of the specification for access instructions.

Lengthy table referenced here
US20260002208A1-20260101-T00116
Please refer to the end of the specification for access instructions.

Lengthy table referenced here
US20260002208A1-20260101-T00117
Please refer to the end of the specification for access instructions.

Lengthy table referenced here
US20260002208A1-20260101-T00118
Please refer to the end of the specification for access instructions.

Lengthy table referenced here
US20260002208A1-20260101-T00119
Please refer to the end of the specification for access instructions.

Lengthy table referenced here
US20260002208A1-20260101-T00120
Please refer to the end of the specification for access instructions.

Lengthy table referenced here
US20260002208A1-20260101-T00121
Please refer to the end of the specification for access instructions.

Lengthy table referenced here
US20260002208A1-20260101-T00122
Please refer to the end of the specification for access instructions.

Lengthy table referenced here
US20260002208A1-20260101-T00123
Please refer to the end of the specification for access instructions.

Lengthy table referenced here
US20260002208A1-20260101-T00124
Please refer to the end of the specification for access instructions.

Lengthy table referenced here
US20260002208A1-20260101-T00125
Please refer to the end of the specification for access instructions.

Lengthy table referenced here
US20260002208A1-20260101-T00126
Please refer to the end of the specification for access instructions.

Lengthy table referenced here
US20260002208A1-20260101-T00127
Please refer to the end of the specification for access instructions.

Lengthy table referenced here
US20260002208A1-20260101-T00128
Please refer to the end of the specification for access instructions.

Lengthy table referenced here
US20260002208A1-20260101-T00129
Please refer to the end of the specification for access instructions.

Lengthy table referenced here
US20260002208A1-20260101-T00130
Please refer to the end of the specification for access instructions.

Lengthy table referenced here
US20260002208A1-20260101-T00131
Please refer to the end of the specification for access instructions.

Lengthy table referenced here
US20260002208A1-20260101-T00132
Please refer to the end of the specification for access instructions.

Lengthy table referenced here
US20260002208A1-20260101-T00133
Please refer to the end of the specification for access instructions.

Lengthy table referenced here
US20260002208A1-20260101-T00134
Please refer to the end of the specification for access instructions.

Lengthy table referenced here
US20260002208A1-20260101-T00135
Please refer to the end of the specification for access instructions.

Lengthy table referenced here
US20260002208A1-20260101-T00136
Please refer to the end of the specification for access instructions.

Lengthy table referenced here
US20260002208A1-20260101-T00137
Please refer to the end of the specification for access instructions.

Lengthy table referenced here
US20260002208A1-20260101-T00138
Please refer to the end of the specification for access instructions.

Lengthy table referenced here
US20260002208A1-20260101-T00139
Please refer to the end of the specification for access instructions.

Lengthy table referenced here
US20260002208A1-20260101-T00140
Please refer to the end of the specification for access instructions.

Lengthy table referenced here
US20260002208A1-20260101-T00141
Please refer to the end of the specification for access instructions.

Lengthy table referenced here
US20260002208A1-20260101-T00142
Please refer to the end of the specification for access instructions.

Lengthy table referenced here
US20260002208A1-20260101-T00143
Please refer to the end of the specification for access instructions.

Lengthy table referenced here
US20260002208A1-20260101-T00144
Please refer to the end of the specification for access instructions.

Lengthy table referenced here
US20260002208A1-20260101-T00145
Please refer to the end of the specification for access instructions.

Lengthy table referenced here
US20260002208A1-20260101-T00146
Please refer to the end of the specification for access instructions.

Lengthy table referenced here
US20260002208A1-20260101-T00147
Please refer to the end of the specification for access instructions.

Lengthy table referenced here
US20260002208A1-20260101-T00148
Please refer to the end of the specification for access instructions.

Lengthy table referenced here
US20260002208A1-20260101-T00149
Please refer to the end of the specification for access instructions.

Lengthy table referenced here
US20260002208A1-20260101-T00150
Please refer to the end of the specification for access instructions.

Lengthy table referenced here
US20260002208A1-20260101-T00151
Please refer to the end of the specification for access instructions.

Lengthy table referenced here
US20260002208A1-20260101-T00152
Please refer to the end of the specification for access instructions.

Lengthy table referenced here
US20260002208A1-20260101-T00153
Please refer to the end of the specification for access instructions.

Lengthy table referenced here
US20260002208A1-20260101-T00154
Please refer to the end of the specification for access instructions.

Lengthy table referenced here
US20260002208A1-20260101-T00155
Please refer to the end of the specification for access instructions.

Lengthy table referenced here
US20260002208A1-20260101-T00156
Please refer to the end of the specification for access instructions.

Lengthy table referenced here
US20260002208A1-20260101-T00157
Please refer to the end of the specification for access instructions.

Lengthy table referenced here
US20260002208A1-20260101-T00158
Please refer to the end of the specification for access instructions.

Lengthy table referenced here
US20260002208A1-20260101-T00159
Please refer to the end of the specification for access instructions.

Lengthy table referenced here
US20260002208A1-20260101-T00160
Please refer to the end of the specification for access instructions.

Lengthy table referenced here
US20260002208A1-20260101-T00161
Please refer to the end of the specification for access instructions.

Lengthy table referenced here
US20260002208A1-20260101-T00162
Please refer to the end of the specification for access instructions.

Lengthy table referenced here
US20260002208A1-20260101-T00163
Please refer to the end of the specification for access instructions.

Lengthy table referenced here
US20260002208A1-20260101-T00164
Please refer to the end of the specification for access instructions.

Lengthy table referenced here
US20260002208A1-20260101-T00165
Please refer to the end of the specification for access instructions.

Lengthy table referenced here
US20260002208A1-20260101-T00166
Please refer to the end of the specification for access instructions.

Lengthy table referenced here
US20260002208A1-20260101-T00167
Please refer to the end of the specification for access instructions.

Lengthy table referenced here
US20260002208A1-20260101-T00168
Please refer to the end of the specification for access instructions.

Lengthy table referenced here
US20260002208A1-20260101-T00169
Please refer to the end of the specification for access instructions.

Lengthy table referenced here
US20260002208A1-20260101-T00170
Please refer to the end of the specification for access instructions.

Lengthy table referenced here
US20260002208A1-20260101-T00171
Please refer to the end of the specification for access instructions.

Lengthy table referenced here
US20260002208A1-20260101-T00172
Please refer to the end of the specification for access instructions.

Lengthy table referenced here
US20260002208A1-20260101-T00173
Please refer to the end of the specification for access instructions.

Lengthy table referenced here
US20260002208A1-20260101-T00174
Please refer to the end of the specification for access instructions.

Lengthy table referenced here
US20260002208A1-20260101-T00175
Please refer to the end of the specification for access instructions.

Lengthy table referenced here
US20260002208A1-20260101-T00176
Please refer to the end of the specification for access instructions.

Lengthy table referenced here
US20260002208A1-20260101-T00177
Please refer to the end of the specification for access instructions.

Lengthy table referenced here
US20260002208A1-20260101-T00178
Please refer to the end of the specification for access instructions.

Lengthy table referenced here
US20260002208A1-20260101-T00179
Please refer to the end of the specification for access instructions.

Lengthy table referenced here
US20260002208A1-20260101-T00180
Please refer to the end of the specification for access instructions.

Lengthy table referenced here
US20260002208A1-20260101-T00181
Please refer to the end of the specification for access instructions.

Lengthy table referenced here
US20260002208A1-20260101-T00182
Please refer to the end of the specification for access instructions.

Lengthy table referenced here
US20260002208A1-20260101-T00183
Please refer to the end of the specification for access instructions.

Lengthy table referenced here
US20260002208A1-20260101-T00184
Please refer to the end of the specification for access instructions.

Lengthy table referenced here
US20260002208A1-20260101-T00185
Please refer to the end of the specification for access instructions.

Lengthy table referenced here
US20260002208A1-20260101-T00186
Please refer to the end of the specification for access instructions.

Lengthy table referenced here
US20260002208A1-20260101-T00187
Please refer to the end of the specification for access instructions.

Lengthy table referenced here
US20260002208A1-20260101-T00188
Please refer to the end of the specification for access instructions.

Lengthy table referenced here
US20260002208A1-20260101-T00189
Please refer to the end of the specification for access instructions.

Lengthy table referenced here
US20260002208A1-20260101-T00190
Please refer to the end of the specification for access instructions.

Lengthy table referenced here
US20260002208A1-20260101-T00191
Please refer to the end of the specification for access instructions.

Lengthy table referenced here
US20260002208A1-20260101-T00192
Please refer to the end of the specification for access instructions.

Lengthy table referenced here
US20260002208A1-20260101-T00193
Please refer to the end of the specification for access instructions.

Lengthy table referenced here
US20260002208A1-20260101-T00194
Please refer to the end of the specification for access instructions.

Lengthy table referenced here
US20260002208A1-20260101-T00195
Please refer to the end of the specification for access instructions.

Lengthy table referenced here
US20260002208A1-20260101-T00196
Please refer to the end of the specification for access instructions.

Lengthy table referenced here
US20260002208A1-20260101-T00197
Please refer to the end of the specification for access instructions.

Lengthy table referenced here
US20260002208A1-20260101-T00198
Please refer to the end of the specification for access instructions.

Lengthy table referenced here
US20260002208A1-20260101-T00199
Please refer to the end of the specification for access instructions.

Lengthy table referenced here
US20260002208A1-20260101-T00200
Please refer to the end of the specification for access instructions.

Lengthy table referenced here
US20260002208A1-20260101-T00201
Please refer to the end of the specification for access instructions.

Lengthy table referenced here
US20260002208A1-20260101-T00202
Please refer to the end of the specification for access instructions.

Lengthy table referenced here
US20260002208A1-20260101-T00203
Please refer to the end of the specification for access instructions.

Lengthy table referenced here
US20260002208A1-20260101-T00204
Please refer to the end of the specification for access instructions.

Lengthy table referenced here
US20260002208A1-20260101-T00205
Please refer to the end of the specification for access instructions.

Lengthy table referenced here
US20260002208A1-20260101-T00206
Please refer to the end of the specification for access instructions.

Lengthy table referenced here
US20260002208A1-20260101-T00207
Please refer to the end of the specification for access instructions.

Lengthy table referenced here
US20260002208A1-20260101-T00208
Please refer to the end of the specification for access instructions.

Lengthy table referenced here
US20260002208A1-20260101-T00209
Please refer to the end of the specification for access instructions.

Lengthy table referenced here
US20260002208A1-20260101-T00210
Please refer to the end of the specification for access instructions.

Lengthy table referenced here
US20260002208A1-20260101-T00211
Please refer to the end of the specification for access instructions.

Lengthy table referenced here
US20260002208A1-20260101-T00212
Please refer to the end of the specification for access instructions.

Lengthy table referenced here
US20260002208A1-20260101-T00213
Please refer to the end of the specification for access instructions.

Lengthy table referenced here
US20260002208A1-20260101-T00214
Please refer to the end of the specification for access instructions.

Lengthy table referenced here
US20260002208A1-20260101-T00215
Please refer to the end of the specification for access instructions.

Lengthy table referenced here
US20260002208A1-20260101-T00216
Please refer to the end of the specification for access instructions.

Lengthy table referenced here
US20260002208A1-20260101-T00217
Please refer to the end of the specification for access instructions.

Lengthy table referenced here
US20260002208A1-20260101-T00218
Please refer to the end of the specification for access instructions.

Lengthy table referenced here
US20260002208A1-20260101-T00219
Please refer to the end of the specification for access instructions.

Lengthy table referenced here
US20260002208A1-20260101-T00220
Please refer to the end of the specification for access instructions.

Lengthy table referenced here
US20260002208A1-20260101-T00221
Please refer to the end of the specification for access instructions.

Lengthy table referenced here
US20260002208A1-20260101-T00222
Please refer to the end of the specification for access instructions.

Lengthy table referenced here
US20260002208A1-20260101-T00223
Please refer to the end of the specification for access instructions.

Lengthy table referenced here
US20260002208A1-20260101-T00224
Please refer to the end of the specification for access instructions.

Lengthy table referenced here
US20260002208A1-20260101-T00225
Please refer to the end of the specification for access instructions.

Lengthy table referenced here
US20260002208A1-20260101-T00226
Please refer to the end of the specification for access instructions.

Lengthy table referenced here
US20260002208A1-20260101-T00227
Please refer to the end of the specification for access instructions.

Lengthy table referenced here
US20260002208A1-20260101-T00228
Please refer to the end of the specification for access instructions.

Lengthy table referenced here
US20260002208A1-20260101-T00229
Please refer to the end of the specification for access instructions.

Lengthy table referenced here
US20260002208A1-20260101-T00230
Please refer to the end of the specification for access instructions.

Lengthy table referenced here
US20260002208A1-20260101-T00231
Please refer to the end of the specification for access instructions.

Lengthy table referenced here
US20260002208A1-20260101-T00232
Please refer to the end of the specification for access instructions.

Lengthy table referenced here
US20260002208A1-20260101-T00233
Please refer to the end of the specification for access instructions.

Lengthy table referenced here
US20260002208A1-20260101-T00234
Please refer to the end of the specification for access instructions.

Lengthy table referenced here
US20260002208A1-20260101-T00235
Please refer to the end of the specification for access instructions.

Lengthy table referenced here
US20260002208A1-20260101-T00236
Please refer to the end of the specification for access instructions.

Lengthy table referenced here
US20260002208A1-20260101-T00237
Please refer to the end of the specification for access instructions.

Lengthy table referenced here
US20260002208A1-20260101-T00238
Please refer to the end of the specification for access instructions.

Lengthy table referenced here
US20260002208A1-20260101-T00239
Please refer to the end of the specification for access instructions.

Lengthy table referenced here
US20260002208A1-20260101-T00240
Please refer to the end of the specification for access instructions.

Lengthy table referenced here
US20260002208A1-20260101-T00241
Please refer to the end of the specification for access instructions.

Lengthy table referenced here
US20260002208A1-20260101-T00242
Please refer to the end of the specification for access instructions.

Lengthy table referenced here
US20260002208A1-20260101-T00243
Please refer to the end of the specification for access instructions.

Lengthy table referenced here
US20260002208A1-20260101-T00244
Please refer to the end of the specification for access instructions.

Lengthy table referenced here
US20260002208A1-20260101-T00245
Please refer to the end of the specification for access instructions.

Lengthy table referenced here
US20260002208A1-20260101-T00246
Please refer to the end of the specification for access instructions.

LENGTHY TABLES
The patent application contains a lengthy table section. A copy of the table is available in electronic form from the USPTO web site (https://seqdata.uspto.gov/docdetail?docId=US20260002208A1). An electronic copy of the table will also be available from the USPTO upon request and payment of the fee set forth in 37 CFR 1.19(b)(3).

1. A method of detecting prognosis for coronavirus infection in an individual, comprising determining the presence or absence of one or more chromosome interactions represented by the probes shown in Table 1 or 3, to thereby determine said prognosis in the individual.

2. A method according to claim 1 wherein:
(i) at least 5 chromosome interactions are typed from Table 1, and/or
(ii) at least 5 chromosome interactions are typed from Table 3.

3. A method according to claim 1 wherein said coronavirus infection is Covid-19 infection.

4. A method according to claim 1 wherein:
(i) at least 5 chromosome interactions are typed from Table 2, and/or
(ii) at least 5 chromosome interactions are typed from Table 4.

5. A method according to claim 1 wherein the chromosome interactions are typed:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, and/or
by a process which detects the proximity of the chromosome regions which have come together in the chromosome interaction.

6. A method according to claim 1 wherein said detecting of the presence or absence of the chromosome interactions is by a process comprising:
(i) in vitro crosslinking of epigenetic chromosomal interactions which are present;
(ii) optionally isolating the cross-linked DNA;
(iii) subjecting said cross-linked DNA to cleaving;
(iv) ligating said cross-linked cleaved DNA ends to form ligated DNA; and
(v) identifying the presence or absence of said ligated DNA;
to thereby determine the presence or absence of the chromosome interaction.

7. A method according to claim 5 wherein said ligated DNA is detected by PCR or by use of a probe.

8. A method according to claim 7 wherein:
(i) detection is by use of a probe, wherein said probe has at least 70% identity to any of the probes shown in Table 1 or 3, or
(ii) detection is by use of PCR, wherein the PCR uses a primer pair that has at least 70% identity to any of the primer pairs shown in Table 1 or 3.

9. A method according to claim 1 wherein:
(i) the method is carried out prognostically, in advance, to detect a high-risk of subsequent severe hyperinflammatory complications for an individual upon exposure to a coronavirus, such as Covid-19, and/or
(ii) the method is carried out to select an individual for receiving therapy or a treatment for coronavirus infection, and/or
(iii) the method is carried out on individual that has been preselected based on a physical characteristic, risk factor or the presence of a symptom, and/or
(iv) the method is carried out to determine prognosis for severity of coronavirus infection, and/or
(v) the method is carried out to determine prognosis for developing sepsis as part of coronavirus disease, and/or
(vi) the method is carried out to determine prognosis for cytokine release syndrome as part of coronavirus disease.

10. A method according to claim 1 wherein the individual:
(i) is suspected of having coronavirus or Covid-19 infection, and/or
(ii) has been admitted to hospital.

11.-12. (canceled)

13. A method of detecting prognosis for coronavirus infection in an individual, comprising determining the presence or absence of one or more chromosome interactions represented by the probes shown in Table 7, to thereby determine said prognosis in the individual.

14. A method according to claim 13 wherein at least 3, 4, 5 or 6 chromosome interactions are typed from Table 7 and/or wherein said coronavirus infection is Covid-19 infection.

15. A method according to claim 13 wherein the chromosome interactions are typed:
in a sample from an individual, and/or
by detecting the presence or absence of a DNA loop at the site of the chromosome interactions, and/or
detecting the presence or absence of distal regions of a chromosome being brought together in a chromosome conformation, and/or
by detecting the presence of a ligated nucleic acid which is generated during said typing and whose sequence comprises two regions each corresponding to the regions of the chromosome which come together in the chromosome interaction, and/or
by a process which detects the proximity of the chromosome regions which have come together in the chromosome interaction.

16. A method according to claim 13 wherein said detecting of the presence or absence of the chromosome interactions is by a process comprising:
(i) in vitro crosslinking of epigenetic chromosomal interactions which are present;
(ii) optionally isolating the cross-linked DNA;
(iii) subjecting said cross-linked DNA to cleaving;
(iv) ligating said cross-linked cleaved DNA ends to form ligated DNA; and
(v) identifying the presence or absence of said ligated DNA;
to thereby determine the presence or absence of the chromosome interaction.

17. A method according to claim 13 wherein said ligated DNA is detected by PCR or by use of a probe.

18. A method according to claim 17 wherein:
(i) detection is by use of a probe, wherein said probe has at least 70% identity to any of the probes shown in Table 7, or
(ii) detection is by use of PCR, wherein the PCR uses a primer pair that has at least 70% identity to any of the primer pairs shown in Table 7.

19. A method according to any claim 17 wherein said ligated DNA is detected using a qPCR system in which the probe has at least 70% identity to any of the probes shown in Table 7 and the primer pairs have at least 70% identity to any of the primer pairs shown in Table 7; wherein preferably the probe sequence is or comprises the sequence of a probe sequence shown in Table 7 and/or each primer has or comprises the sequence of a primer shown in Table 7.

20. A method according to claim 17 wherein the presence or absence of all 6 of the chromosome interactions represented by the probes of Table 7 is detected, optionally using all the probes and/or all primer pairs shown in Table 7.

21. A method according to claim 13 wherein:
(i) the method is carried out prognostically, in advance, to detect a high-risk of subsequent severe hyperinflammatory complications for an individual upon exposure to a coronavirus, such as Covid-19, and/or
(ii) the method is carried out to select an individual for receiving therapy or a treatment for coronavirus infection, and/or
(iii) the method is carried out on individual that has been preselected based on a physical characteristic, risk factor or the presence of a symptom, and/or
(iv) the method is carried out to determine prognosis for severity of coronavirus infection, and/or
(v) the method is carried out to determine prognosis for developing sepsis as part of coronavirus disease, and/or
(vi) the method is carried out to determine prognosis for cytokine release syndrome as part of coronavirus disease.

22. A method according to claim 13 wherein the individual:
(i) is suspected of having coronavirus or Covid-19 infection, and/or
(ii) has been admitted to hospital.

23. A method according to claim 1, wherein the typing of chromosome interactions comprises specific detection of the ligated product by quantitative PCR (qPCR) which uses primers capable of amplifying the ligated product and a probe which binds the ligation site during the PCR reaction, wherein said probe comprises sequence which is complementary to sequence from each of the chromosome regions that have come together in the chromosome interaction, wherein preferably said probe comprises:
an oligonucleotide which specifically binds to said ligated product, and/or
a fluorophore covalently attached to the 5′ end of the oligonucleotide, and/or
a quencher covalently attached to the 3′ end of the oligonucleotide, and
optionally
said fluorophore is selected from HEX, Texas Red and FAM; and/or
said probe comprises a nucleic acid sequence of length 10 to 40 nucleotide bases, preferably a length of 20 to 30 nucleotide bases.

24. A method according to claim 1 comprising determining the presence or absence of one or more chromosome interactions represented by the probes shown in any of Tables 8, 9, 10, 11, 12, 13, 14, 15, 16 or 17, to thereby determine said prognosis in the individual.

25. A method according to claim 1 comprising determining the presence or absence of:
one or more chromosome interactions associated with a severe coronavirus infection, wherein preferably said chromosome interactions are shown in Table 8, 9, 14 or 15 and/or
one or more chromosome interactions associated with a mild coronavirus infection, wherein preferably said chromosome interactions are shown in Table 10, 11, 16 or 17.

26. A method according to claim 19 wherein
(i) detection is by use of a probe, wherein said probe has at least 70% identity to any of the probes shown in Table 9 or 11, or
(ii) detection is by use of PCR, wherein the PCR uses a primer pair that has at least 70% identity to any of the primer pairs shown in Table 9 or 11.

27. A method according to claim 26 wherein said ligated DNA is detected using a qPCR system in which the probe has at least 70% identity to any of the probes shown in Table 9, 11, 14 or 16 and the primer pairs have at least 70% identity to any of the primer pairs shown in Table 9, 11, 14 or 16; wherein preferably the probe sequence is or comprises the sequence of a probe sequence shown in Table 9, 11, 14 or 16 and/or each primer has or comprises the sequence of a primer shown in Table 9, 11, 14 or 16.

28. A method according to claim 1 wherein the presence or absence of at least 5 of the chromosome interactions represented by the probes of Table 9 or 11 is detected, optionally using all the probes and/or all primer pairs shown in Table 9 or 11.

29. A method according to claim 1 which comprises detecting the sepsis status of the individual by detecting the presence or absence of any of the chromosome interactions shown in Tables 12 or 13.

30. A method according to claim 1 for treatment of severe coronavirus disease, said method comprising:
identifying whether an individual is susceptible to severe coronavirus disease by the method of claim 1, and
administering to any individual identified as being susceptible a therapeutic agent selected from any of the agents shown in Table 6.

31. A method according to claim 1 which:
prevents or treats coronavirus infection, and/or
prevents or treats a detrimental immune response; comprising
identifying an individual as being susceptible to severe disease as a result of coronavirus infection by the method of claim 1, and
administering to the identified individual an agent which prevents or treats coronavirus infection and/or prevents or treats a detrimental immune response.

32. A method according to claim 29 for treatment of sepsis, said method comprising:
identifying whether an individual is susceptible to or has sepsis by the method of claim 29, and
administering an agent which treats sepsis to an individual identified as being susceptible to or having sepsis.

33. (canceled)
